(19) United States
(12) Patent Application Publication
(10) Pub. No.: US 2003/0167129 A1
(43) Pub. Date: Sep. 4, 2003

(54) Binding compounds and methods for identifying binding compounds

(76) Inventors: John J. Nestor JR., Bedford, MA (US); Carol J. Wilson, Somerville, MA (US); Christina A. Tan Hehir, Arlington, MA (US); Steven A. Kates, Needham, MA (US); John Krstenansky, Belmont, MA (US)

Correspondence Address: John Schulte, Consensus Pharmaceuticals, Inc., 200 Boston Avenue, Medford, MA, 02155, US

(21) Appl. No.: 10/014,322

(22) Filed: Oct. 26, 2001

Related U.S. Application Data
(63) Continuation-in-part of application No. 09/813,651, filed on Mar. 20, 2001.
(60) Provisional application No. 60/243,587, filed on Oct. 27, 2000.

(51) Int. Cl. C12Q 1/70; G01N 33/53; G06F 19/00; G01N 33/48; G01N 33/50; G01N 33/543

(52) U.S. Cl. 702/19; 435/7.1; 435/5; 436/518

(57) ABSTRACT

Binding compounds for CXC chemokine receptor 4 and methods for identifying binding compounds for CXC chemokine receptor 4 are provided. Also provided are therapeutic agents comprising such compounds.

### CROSS REFERENCE TO RELATED APPLICATIONS

This application claims the benefit of U.S. S. No. 60/243,587, filed Oct. 27, 2000, and U.S. Ser. No. 09/813,651, filed Mar. 20, 2001, the disclosure of each of which is incorporated by reference herein and further claims the benefit of U.S. Ser. No. 09/813,653 and U.S. Ser. No. 09/813,448 both filed on Mar. 20, 2001, the disclosure of each of which is incorporated by reference herein.

### GOVERNMENT LICENSE RIGHTS

Certain work described herein was supported, in part, by Federal Grant No. R1-R44-AI50414-01, awarded by the National Institutes of Health. The Government may have certain rights in the invention.

### FIELD OF THE INVENTION

The invention generally relates to Cysteine-X-Cysteine Chemokine Receptor 4 (“CXCR4” or “CXC chemokine receptor 4”), and more particularly, to binding compounds for CXC chemokine receptor 4. Methods of the invention are useful for the treatment of disease by identifying and preparing binding compounds for CXC chemokine receptor 4.

### BACKGROUND OF THE INVENTION

Chemokines (chemoattractant cytokines) comprise a family of structurally related secreted proteins of about 70-110 amino acids that share the ability to induce migration and activation of specific types of blood cells. See Proost P., et al. (1996) Int. J. Clin. Lab. Rse. 26: 211-223; Premack, et al. (1996) Nature Medicine 2: 1174-1178; Yoshie, et al. (1997) J. Leukocyte Biol. 62: 634-644. Over 30 different human chemokines have been described to date. While they are primarily responsible for the activation and recruitment of leukocytes, they vary in their specificities for different leukocyte types (neutrophils, monocytes, eosinophils, basophils, lymphocytes, dendritic cells, etc.), and in the types of cells and tissues where the chemokines are synthesized. Further analysis of this family of proteins has shown that it can be divided up into two further subfamilies of proteins. These have been termed CXC or α-chemokines, and the CC or β-chemokines based on the spacings of two conserved cysteine residues near the amino terminus of the proteins.

Chemokines are typically produced at sites of tissue injury or stress, where they promote the infiltration of leukocytes into tissues and facilitate an inflammatory response. Some chemokines act selectively on immune system cells such as subsets of T-cells or B lymphocytes or antigen presenting cells, and may thereby promote immune responses to antigens. In addition, some chemokines have the ability to regulate the growth or migration of hematopoietic progenitor and stem cells that normally differentiate into specific leukocyte types, thereby regulating leukocyte numbers in the blood.

The activities of chemokines are mediated by cell surface receptors that are members of a family of seven transmembrane (“7TM”), G-protein coupled receptors (“GPCR”). At least twelve different human chemokine receptors are known, including CCR1, CCR2, CCR3, CCR4, CCR5, CCR6, CCR7, CCR8, CXCR1, CXCR2, CXCR3, and CXCR4. These receptors vary in their specificities for specific chemokines. Some receptors bind to a single known chemokine, while others bind to multiple chemokines. Binding of a chemokine to its receptor typically induces intracellular signaling responses such as a transient rise in cytosolic calcium concentration, followed by cellular biological responses such as chemotaxis. In addition, some chemokine receptors, such as CXCR4, serve as co-receptors for Human Immunodeficiency Virus (HIV), such that they interact with HIV and with the cellular CD4 receptor to facilitate viral entry into cells.

Chemokines are important in medicine because they regulate the movement and biological activities of leukocytes in many disease situations, including, but not limited to: allergic disorders, autoimmune diseases, ischemia/reperfusion injury, development of atherosclerotic plaques, cancer (including mobilization of hematopoietic stem cells for use in chemotherapy or myeloprotection during chemotherapy), chronic inflammatory disorders, chronic rejection of transplanted organs or tissue grafts, chronic myelogenous leukemia, and infection by HIV and other pathogens Furthermore, CXCR4, in particular, has been implicated in diseases such as glioblastoma multiforme tumor, hepatocellular carcinoma, colon cancer, esophageal cancer, gastric cancers, breast cancer metastasis, pancreatic cancer and in renal allograft rejection. See e.g., Sehgal A, et. al., J. Surg. Oncol. 69(2)₉₉-104 (1998); Begum NA, et. al., Int. J. Oncol. 14(5)927-934 (1999); Mitra P, et. al., Int. J. Oncol. 14(5):917-25 (1999); Muller A, et. al., Nature 410(6824)50-6 (2001); Koshiba T, et. al., Clin. Cancer Res. 6(9):3530-5 (2000); and Eitner F, et. al., Transplantation 66(11):1551-7 (1998).

Antagonists of chemokine receptors may be of benefit in many of these diseases by reducing excessive inflammation and immune system responses. In the case of HIV infection, chemokines and antagonists that bind to HIV co-receptors may have utility in inhibiting viral entry into cells. HIV causes Acquired Immune Deficiency Syndrome (“AIDS”), which is one of the leading causes of death in the United States and throughout the world. According to the Center for Disease Control, at least 30.6 million people world-wide have been infected with HIV. HIV attacks the immune system and leaves the body vulnerable to a variety of life-threatening illnesses. Common bacteria, yeast, and viruses that would not cause disease in people with a fully functional immune system often cause these illnesses in people affected with HIV.

Not all patients infected with HIV have AIDS. Typically, a patient who has been infected with HIV will slowly develop AIDS as HIV damages his immune system. The severity of the immune system damage is measured by an absolute CD4⁺ lymphocyte count; a patient having a count of less than 200 cells/μl is considered to have AIDS. The CD4 protein is a glycoprotein of approximately 60,000 molecular weight and is expressed on the cell membrane of mature, thymus-derived (T) lymphocytes, and to a lesser extent on cells of the monocyte/macrophage lineage. Typically, CD4 cells appear to function by providing an activating signal to B cells, by inducing T lymphocytes bearing the reciprocal CD8 marker to become cytotoxic/suppressor cells, and/or by interacting with targets bearing major histocompatibility complex (MHC) class II molecules.

The search for a preventative or therapeutic agent for HIV and AIDS has been especially intense as this epidemic has proliferated world-wide. Research has discovered that the ability of HIV to enter cells requires the binding of the HIV envelope glycoproteins encoded by the env gene to the CD4 receptor. These glycoproteins are encoded by the env gene and translated as a precursor, gp160, which is subsequently cleaved into gp120 and gp41. Gp120 binds to the CD4 protein present on the surface of susceptible target cells, resulting in the fusion of virus with the cell membranes, and facilitating virus entry into the host. The eventual expression of env on the surface of the HIV-infected host cell enables this cell to fuse with uninfected, CD4⁺ cells, thereby spreading the virus. However, in response to infection with HIV, the host immune system will produce antibodies targeted against various antigenic sites, or determinants, of gp120. Some of those antibodies will have a neutralizing effect and will inhibit HIV infectivity. It is believed that this neutralizing effect is due to the antibodies' ability to interfere with HIV's cellular attachment. It is also believed that this effect may explain in part, the rather long latency period between the initial seroconversion and the onset of clinical symptoms.

Recent studies have shown that the HIV fusion process occurs with a wide range of human cell types that either express human CD4 endogenously or have been engineered to express human CD4. The fusion process, however, does not occur with nonhuman cell types engineered to express human CD4. Although such nonhuman cells can still bind env, membrane fusion does not follow. The disparity between human and nonhuman cell types exists apparently because membrane fusion requires the co-expression of human CD4 and a co-receptor specific to human cell types. Because they lack this co-receptor accessory factor, nonhuman cell types engineered to express only human CD4 are incapable of membrane fusion, and are thus nonpermissive for HIV infection. Furthermore, expression of CD4 in some human cell lines was insufficient to confer resistance to HIV-1 infection. In addition, some HIV-1 strains were T cell tropic (T-tropic) while others were macrophage tropic (M-tropic), though both cells possessed the CD4 antigen. Further research has shown that certain chemokines could block the infectivity of M-tropic but not T-tropic HIV strains. Thereafter, it was shown that an orphan receptor, CXCR4 was required for the activity of T-tropic strains. See e.g., Horuk R., Immunol Today 20(2):89-94 (1999); Doms R W, Peiper S C., Virology 235(2):179-90 (1997); Ward S G, Bacon K, Westwick J., Immunity 9(1):1-11 (1998); Berson J F, Doms R W., Semin Immunol 10(3):237-48 (1998).

While it has been demonstrated that HIV uses the CXCR4 as a co-receptor for cellular entry, it has been difficult for researchers to obtain high resolution X-ray crystallographic structures of a CXCR4 because of difficulties in crystallizing such a 7TM protein which requires complex interactions with lipids for its native conformation. The requirement of the interaction with lipids also makes difficult the preparation of biologically active forms of such GPCRs, because, in the absence of those lipids, they readily form denatured aggregates with minimal to no ability to specifically bind ligands unless great care is taken to preserve the biologically active conformation during solubilization. In the absence of an X-ray structure, a variety of approaches have been used to define the regions of CXCR4 that are involved in gp120 binding and viral uptake. These approaches generally involve comparing results with non-human homologues, chimeric receptors, and point mutants to study the structural requirements for the co-receptor activity of CXCR4. CXCR4, is a 352 amino acid protein, has seven putative transmembrane (“TM”) segments (TM1=residues 40-64, TM2=77-99, TM3=111-131, TM4=177-197, TM5=204-223, TM6=241-261, and TM7=283-307, putatively), and extracellular N-terminus, three extracellular loops and three intracellular loops connecting the transmembrane segments, and an intracellular C-terminus. The second extracellular loop is the region most required for the entry of HIV into the cell; however, the N-terminus and the third extracellular loop are also involved. Several charged residues on the extracellular side of the receptor have been implicated in binding (Asp-11, Asp-265, Glu-275, Glu-278, and Arg-280) using mutagenesis studies. See e.g., Zhou H, et. al., Arch. Biochem. Biophys. 373(1):211-7 (2000). In addition, the N-terminus of the ligand SDF-1 has been implicated as important for binding to the receptor. See e.g., Crump M P, et. al., EMBO J. 16(23):6996-7007 (1997). Furthermore, synthetic peptides have been used to study the effect of high positive charge in peptides on the interaction with CXCR4. See e.g., Luo Z, et. al., Biochem. Biophys. Res. Comm. 263:691-5 (1999).

Several inhibitors of CXCR4 have been reported. These include positively charged peptides such as T22 and T140 and small molecule inhibitors such as ALX40-4C and T134. AMD 3100, a heterocyclic bicyclam (one positive charge on each of two rings) has been reported as well. See e.g., Tamamura H, et. al., Bioorg Med Chem 6(7):1033-41(1998); Tamamura H, et. al., Biochem. Biophys. Rs. Comm. 252:877-82 (1998); Doranz B J, et. al., J Exp Med 186(8):1395-400 (1997); Arakaki R, et. al., J. Virol. 73(2):1719-23 (1999); and Donzella G A, et. al., Nat Med 4(1):72-7 (1998). AMD 3100, however, caused the conversion from T- to M-tropic viruses in Peripheral Blood Monocytes (“PBMCs”). See e.g., De Clercq E., Mol. Pharmacol. 57:833-839 (2000). In addition, bicyclams may interfere with other CXCR4-like receptors. See e.g., Schols D, et. al., J. Exp. Med. 186(8):1383-1388 (1997). Furthermore, T22 inhibits calcium mobilization, therefore interfering with CXCR4's natural required signaling. See e.g., Murakami T, et. al., J. Exp. Med. 186(8):1389-1393 (1997).

As a result of the limitations of prior inhibitors of CXCR4 binding, a need still remains for effective HIV preventative and therapeutic agents, and methods for identifying candidates thereof It has been demonstrated that HIV uses the CXCR4 as a co-receptor for cellular entry that can be blocked by its natural ligands and this makes a high affinity ligand for CXCR4 an important therapeutic target. GPCRs in general, and CXCR4 in particular, are very difficult to solubilize and purify because they normally need to fold and be maintained in the presence of the native lipids of the cell membrane. Simple expression and precipitation with antibodies result routinely in denatured aggregates with little or no ability to specifically bind native ligands. Accordingly, there is a need in the art for methods of identifying CXCR4 binding compounds and identification of CXCR4 binding therapeutics with which to prevent or treat diseases such as AIDS. Such therapeutics may comprise peptides, peptidomimetics, or small molecules that can inhibit natural ligand binding to CXCR4. Such methods and compositions are provided herein.

### SUMMARY OF THE INVENTION

The present invention provides binding compounds for CXCR4 and methods for identifying those binding compounds. In one embodiment, screening methods are provided to identify binding motifs for CXCR4, as well as ligands capable of binding to CXCR4. In another embodiment, the invention comprises the design and identification of therapeutic peptides, peptidomimetics, or small molecules suitable for use in the prevention or treatment of HIV and AIDS.

In one embodiment, methods of the invention provide for the synthesis and purification of linear and cyclic peptide libraries useful for screening and identifying a binding motif for CXCR4, as well as screening for potential ligands thereof. Methods of the invention provide for the incorporation of unnatural amino acids and amino acids of the D configuration into linear or cyclic peptides for use in such libraries. Libraries comprising peptides having such amino acids demonstrate enhanced binding affinity and duration of action in vivo resulting from resistance to proteolysis.

In a preferred embodiment, the invention provides for the use of highly diverse libraries of peptide (linear and cyclic, natural and unnatural amino acids), peptidomimetic, and small molecule compounds for the lead ligand identification step. Such ligands may be directly or indirectly agonistic or antagonistic to CXCR4 binding activity.

In a preferred embodiment, the invention provides for the use of phage display methods for the identification of preliminary motif information, followed by additional rounds of affinity purification with purified receptor preparations of the invention and highly diverse libraries. In a particularly preferred embodiment, phage display technology is combined with the use of cyclic peptide and/or peptidomimetic libraries.

In another embodiment of the invention, computer-aided design technology is used to virtually screen, identify, design, or validate lead compounds for agonistic or antagonistic potential with regard to CXCR4 activity. Such technology uses computer-generated, three-dimensional images based upon molecular and structural information of both the CXCR4 and the potential binding partners by virtually aligning the protein with the binding partners. In the case of a library designed for computer-aided screening, a great deal of the information necessary for lead optimization is obtained directly from the library design. In one embodiment, potential leads are identified by prior screening of an actual library or through some other means. One embodiment of the invention involves the screening of biologically appropriate drugs that relies on structure based rational drug design. In such cases, a three dimensional structure of the protein (or similar family member), peptide or molecule is determined and potential agonists and/or antagonists are designed with the aid of computer modeling. In a preferred embodiment of the invention, after an appropriate drug is identified, the drug is contacted with CXCR4, whereby a binding complex is formed between the potential drug and CXCR4. Methods of contacting the drug to CXCR4 are generally understood by anyone having skill in the art of drug development.

In another embodiment, the present invention provides for the use of partially purified CXCR4 receptor protein as the agent for carrying out the selection, identification, and improvement of tight binding ligands in identifying therapeutically useful compounds. In a preferred embodiment, the invention comprises the use of tagging methods to generate a modified CXCR4 receptor protein that functions to facilitate purification and identification steps involved in the screening methods. In another embodiment, the invention comprises a nucleic acid sequence corresponding to the receptor CXCR4 fused to tag sequences (i.e., GST, FLAG, 6×His, dual tagged with FLAG-GST, C-MYC, MBP, V5, Xpress, CBP, HA) with appropriate specific protease sites engineered into the vector.

In a particularly preferred embodiment, methods of the invention provide for solubilization or immobilization of CXCR4 to facilitate ligand selection methods provided herein. CXCR4 may be derived from any source, including without limitation: inactive, precipitated protein preparations; cell membrane preparations; and, whole cell preparations. In one embodiment, the invention provides for a method of screening combinatorial libraries directly for general affinity determination using membranes from baculovirus expression systems or any other appropriate expression system. In one embodiment of the invention, partially purified CXCR4 is used in carrying out the selection, identification, and improvement of tight binding ligands. In a preferred embodiment, partially purified, tagged CXCR4 is used in a sequestered form to screen diverse libraries (focused or highly diverse) for the affinity purification of a tight binding ligand. In a highly preferred embodiment of the invention, the conditions for solubilization or immobilization of the appropriate ligand provide for the use of low salt, such as, for example, low magnesium or calcium concentrations; and no sodium chloride (“NaCl”) (0.0 nM NaCl).

In another embodiment, the invention further comprises the step of eluting bound components of the libraries from the immobilized protein with specific N-terminally blocked peptides or other non-sequencable analogs. In yet another embodiment, the invention comprises the optional step of binding combinatorial libraries to a resin-immobilized protein. In another embodiment, the invention comprises a purified polypeptide with tag sequences, which may be optionally immobilized onto an appropriate affinity resin for assay. A further embodiment comprises the step of releasing or eluting tagged protein with its bound library with specific N-terminally blocked peptides or other non-sequencable analogs. In yet another embodiment, a method of the invention comprises the step of cleaving a tag from a protein of interest using a specific protease (as designed into the protein/vector) after immobilization onto an affinity resin and after the combinatorial library is bound to release the complex.

In yet another embodiment, the target ligand is selected from a linear peptide library, a peptidomimetic library, a cyclic peptide library, or a focused library developed using an initial motif identified by phage display techniques or a library combining any of the foregoing. In another embodiment, a target ligand is eluted from the receptor preparation using a peptide or other ligand, or by using pH change or chaotropic agents, such as urea or guanidine hydrochloride, that can disrupt the hydrogen bonding structure of water and denature proteins in concentrated solutions by reducing the hydrophobic effect. Also contemplated by the invention are ligands for CXCR4 identified using the methods disclosed herein. In yet another embodiment of the invention, protein sequencing techniques are used for the determination of the structure of the ligand identified by the affinity purification step.

In another embodiment, the invention comprises therapeutic agents, such as, for example, a small molecule antagonist of CXCR4 binding that are identified using methods of the invention appropriate for the treatment of a disease or disorder, such as, for example, HIV infection or AIDS. In another embodiment, a patient infected with HIV is treated with a therapeutic agent comprising a compound identified using methods of the invention, or a small molecule antagonist of CXCR4 binding. In another embodiment, a patient infected with HIV is treated through the use of combinations of therapeutics that include, for example, CXCR4 inhibitors and reverse transcriptase and protease inhibitors.

A detailed description of certain preferred embodiments of the invention is provided below. Other embodiments of the invention are apparent upon review of the detailed description that follows.

### DESCRIPTION OF THE DRAWINGS

FIG. 1 shows an exemplary peptide library with a fixed, non-degenerate lysine or arginine and eight degenerate positions consisting of eighteen amino acids in approximately equal proportion.

FIG. 2 shows an exemplary peptide library screening using binding domains.

FIG. 2a shows a SDS-PAGE of cell lysate containing CXCR4 and of purified CXCR4 stained with coomassie.

FIG. 3 shows an isolated human CXCR4 cDNA sequence (SEQ ID NO: 125).

FIG. 4 shows an exemplary baculovirus transfer vector for CXCR4-HIS.

FIG. 5 shows an exemplary baculovirus transfer vector for CXCR4-FLAG.

FIG. 6 shows an exemplary baculovirus transfer vector for CXCR4-GST.

FIG. 7a is a chart showing representative radioligand saturation binding studies using membrane preparations of GST-CXCR4 (High Five).

FIG. 7b is a chart showing representative displacement curves for radioligand binding studies using membrane preparations of GST-CXCR4 (High Five).

FIG. 8 shows the immobilization of GPCRs for affinity purification from libraries.

FIG. 9 depicts representative displacement curves for CXCR4.

FIG. 10 is a bar graph showing representative high-throughput ligand-binding inhibition for CXCR4.

FIG. 11 depicts IC₅₀'s for certain exemplary CXCR4 analogs of the present invention.

FIG. 12 depicts the inhibition of HIV infection by certain exemplary CXCR4 peptide inhibitors of the present invention.

FIG. 13 depicts the inhibition of HIV infection by an exemplary CXCR4 peptide inhibitor of the present invention.

### DETAILED DESCRIPTION OF THE INVENTION

Generally, methods of the invention provide for the determination of a binding motif for CXCR4. Further, methods of the invention provide for the identification of agonists or antagonists of the interaction of CXCR4 with its natural ligand, thereby providing for the identification of therapeutic lead compounds. Methods for library design and synthesis, and library screening that are particularly useful in the invention are described in the following patent and patent applications, the disclosure of each of which is incorporated by reference herein: Cantley et al., U.S. Pat. No. 5,532,167; Cantley, et al., U.S. Ser. No. 08/369,643, filed Dec. 17, 1998; Cantley, et al., U.S. Ser. No. 08/438,673, filed Nov. 12, 1999; Hung-Sen, et al, U.S. Ser. No. 09/086,371, filed May 28, 1998; Hung-Sen, et al., U.S. Ser. No. 08/864,392, filed Jun. 24, 1999; and Lai, et al., U.S. Ser. No. 09/387,590, filed Aug. 31, 1999.

According to the methods of the invention, CXCR4 is cloned and expressed, and tested for activity. The CXCR4 may be tagged on the C-terminus or on the N-terminus to facilitate the determination of the character of the CXCR4's ligand-binding properties. Exemplary tags include, without limitation, 6×His, FLAG, GST, V5, Xpress, c-myc, HA, CBD, and MBP. The tagged CXCR4 is used in screening of libraries comprising, for example, linear and/or cyclic peptides having natural and/or unnatural amino acids, peptidomimetics and/or small molecules. Such peptidomimetics and small molecules may comprise any natural or synthetic compound, composition, chemical, protein, or any combination or modification of any of the foregoing that is used to screen for binding compounds of CXCR4.

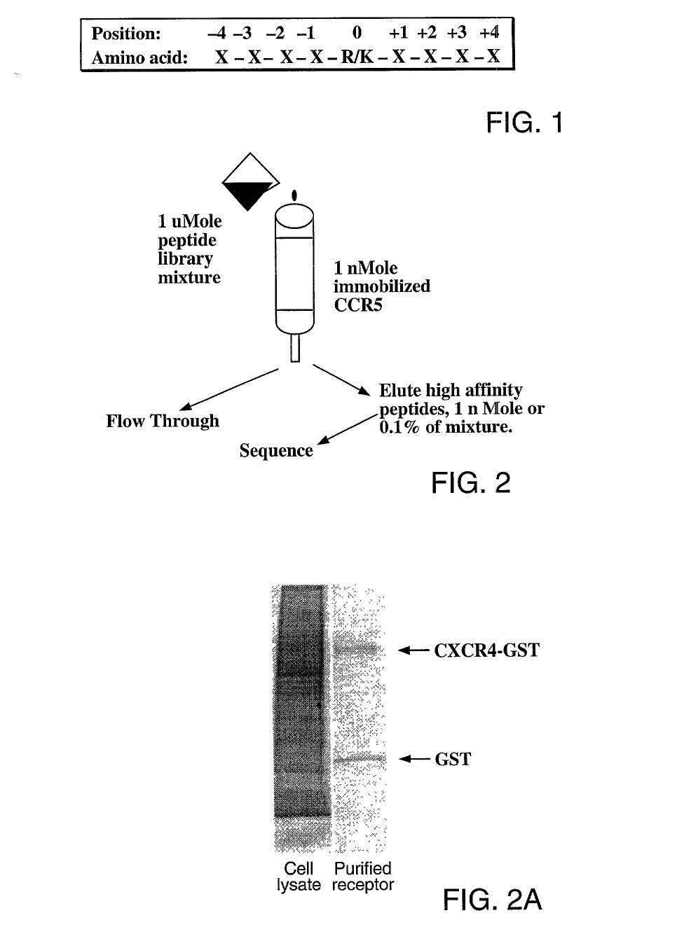

In one aspect, an oriented degenerate peptide library useful in methods of the invention employs soluble peptide libraries consisting of one or more amino acids in non-degenerate positions, known or suspected to be important for ligand binding, and eighteen amino acids in approximately equal proportions in degenerate positions. Cysteine and tryptophan may be omitted to avoid certain analytical difficulties on sequencing. Such a library is shown in FIG. 1, where X represents a degenerate position consisting of any of eighteen amino acids and a lysine or arginine is fixed at a non-degenerate position. Furthermore, the selection of arginine or lysine as an orienting residue is based on the fact that basic residues of gp120 are important determinants in binding to CXCR4. Another aspect of the invention involves the selection of any amino acid as an orienting residue. Additional residues can be added to the N-terminal of the sequence shown in FIG. 1 because there are often interfering substances present in the first and second sequencing cycles. Additional residues can be added at the C-terminal end to provide amino acids to better anchor the peptide to the filter in the sequencer cartridge.

Another aspect of the invention provides for the use of highly diverse libraries of peptide (linear and cyclic, natural and unnatural amino acids), peptidomimetic, and small molecule compounds for the lead identification step. For example, these ligands can be agonistic or antagonistic in their function on the receptor. Generally, the invention uses partially purified CXCR4 as the agent for carrying out the selection, identification, and improvement of tight binding ligands as a route to therapeutically useful compounds. In addition, the invention provides for the development and use of solubilization as well as immobilization procedures that facilitate the efficient ligand selection methods as provided herein. Specifically, the optimal conditions for solubilization and/or immobilization for efficient ligand selection comprise the use of low salt, such as, for example, low or no magnesium or calcium concentrations, and no NaCl concentrations (0.0 nM NaCl). Ligand selection methods using, for example, inactive, precipitated protein, cell membrane preparations, and whole cell preparations are further provided herein.

In one aspect of the invention, the screening step may comprise phage display technology. Such phage display systems have been used to screen peptide libraries for binding to selected target molecules and to display functional proteins with the potential of screening these proteins for desired properties. More recent improvements of the display approach have made it possible to express enzymes as well as antibody fragments on the bacteriophage surface thus allowing for selection of specific properties by selecting with specific ligands. See e.g., Smith S F, et. al., Methods Enzym. 217:228-257 (1993). Phage display methods may be used for the identification of preliminary motif information, and can be followed by additional rounds of affinity purification with purified receptor preparations of the invention and highly diverse libraries, especially cyclic peptide and peptidomimetic libraries. The phage display methods allow the identification of motifs of natural amino acids. Information derived from phage display can be applied to affinity purification methods using, for example, synthetic libraries containing novel amino acid analogs or cyclic peptides to select ligands that have enhanced pharmaceutical characteristics. The use of initial, secondary and tertiary libraries allows a more complete definition of the specificity of the binding site. Secondary libraries may be sequenced incorporating information from the initial library. With the first library, some degenerate positions may yield high preferences for specific amino acids and these may become non-degenerate positions consisting of the preferred amino acid in a second library. See e.g., Wu R, J Biol Chem 271(27):15934-41 (1996).

Alternatively, or in addition, computer-aided design technology may be used in the screening and/or designing of peptides, peptidomimetics, and small molecules. Together with information such as, for example, the crystal structure of rhodopsin (see e.g., Palczewski, et al., Science 289(5480):739-745 (2000)) along with the sequence of CCR5, transmembrane predictions, and any structural information obtained from mutagenesis studies, computer aided design technology may virtually screen, identify, design and validate potential compounds with regards to their CXCR4 activity. Computer programs that may be used to aid in the design of appropriate peptides, peptidomimetics and small molecules include, for example, DOCK (which may be obtained, for example, from University of California, San Francisco), FRODO (which may be obtained, for example, from University of Alberta) and INSIGHT (which may be obtained, for example, from Accelrys, San Diego, Calif.). An example of a method for screening of biologically appropriate drugs relies on structure based rational drug design. In such cases, a three dimensional structure of the protein, peptide or molecule is determined (or modeled after a close family member) and potential agonists and/or antagonists are designed with the aid of computer modeling. See e.g., Butt et al., Scientific American, December 92-98 (1993); West et al., TIPS, 16:67-74 (1995); Dunbrack et al., Folding & Design, 2:27-42 (1997). After an appropriate drug is identified, the drug is contacted with CXCR4, wherein a binding complex forms between the potential drug and CXCR4. Methods of contacting the drug to CXCR4 are generally understood by anyone having skill in the art of drug development.

The screening step may be performed in solution phase, or with the CXCR4 immobilized on affinity columns. In addition to the immobilization of tagged CXCR4 using an affinity resin, other forms of sequestration can be used to perform the affinity purification of select ligands from libraries. These include, but are not limited to the following examples. The receptor and bound library components can be separated from non-bound library components using equilibrium dialysis. The tagged receptor can be bound to specific affinity membranes, which are in the form of plates or are separate. The libraries can then be incubated with the membrane and easily washed to remove non-specific binding components. Size exclusion methodology can be used to separate a purified receptor bound library complex from unbound components after pre-incubating the receptor with the library. Additionally, a micellar complex containing the receptor (which may or may not incorporate lipids as well as detergent) can be separated after binding select affinity components from a library by differential centrifugation. Generally, the high affinity ligand can be released using low pH or high salt conditions and the structure identified by sequencing as described herein.

In order to determine those ligands that had the highest affinity to the target receptor, generally, over 200 peptide libraries were screened to determine each library's respective inhibition binding. In general, a greater than 10% inhibition at 100 μM was significant for continued evaluation of the sequence via affinity purification. In additional aspects of the invention, once preferred amino acid residues are identified due to high preference values by CXCR4 at the degenerate positions of the library, specific peptides are synthesized by the same methods as employed for library synthesis. In one embodiment of the invention, a high preference value is greater than 1. The value is determined by subtracting the control value from the sample value and dividing by the reference value. In a preferred embodiment of the invention, the preference value is greater than 1.2. In a highly preferred embodiment of the invention, the preference value is greater than 2. After synthesis of the identified peptide sequence, the peptide is purified by, for example, High Performance Liquid Chromatography (“HPLC”) and compositions are confirmed by Matrix-Assisted Laser Desorption Ionization-Time of Flight Mass Spectrometer (“MALDI-TOF MS”) and Edman Sequencing. Generally, relative affinities may be measured by modifying the radiolabel binding assay used in receptor purification.

To further enhance the specificity of the motif obtained from the affinity purified peptides, other methods can be used. The bound components of the libraries can be eluted from the immobilized protein with specific N-terminally blocked peptides or other non-sequencable analogs. To avoid the release of minor contaminants from an affinity resin after binding of the library, the release/elution of the tagged CXCR4 with its bound library can be accomplished using specific N-terminally blocked peptides or other non-sequencable analogs. This can be done using acetylated FLAG peptide to elute CXCR4-FLAG receptor from the resin. Alternatively, the tag from CXCR4 may be cleaved using a specific protease (as designed into the protein/vector; either enterokinase or thrombin) after immobilization onto an affinity resin and after the combinatorial library is bound to release the complex. Finally, libraries can be prescreened for their ability to bind to the receptor (using significantly less protein) by a binding assay using CXCR4-containing membranes from, for example, Sf9 (Spodoptera frugiperda) or High Five (Trichoplusia ni) cells (both obtained from Invitrogen, Carlsbard, Calif.) in a single assay or in an array assay. This screening may be performed using CXCR4 and a number of linear and cyclic libraries to determine their effectiveness in inhibiting the natural ligand to bind.

Methods of the invention further comprise the design of therapeutic agents comprising peptides, peptidomimetics, and/or small molecules that are antagonistic to CXCR4 activity appropriate for the treatment of patients with a disease, such as AIDS. Binding compounds for CXCR4 and the identification of optimal synthesis and purification thereof provides for an effective treatment of AIDS and HIV infection. For example, the small peptide ligand binding compounds of the invention, both cyclic and linear peptide ligands, demonstrate enhanced binding affinity and anti-viral activity, and are resistant to proteolysis as identified, for example, in Table 1. Amino acids and peptides are abbreviated and designated following the rules of the IUPAC-IUB Commission of Biochemical Nomenclature in J. Biol. Chem. 247, 977-983 (1972). Amino acid symbols denote the L-configuration unless indicated otherwise.

In general, amino acids from the fragments of gp120 determined to be crucial for viral uptake have been used to specify fixed, or non-degenerate positions in the peptide libraries that have been designed for use in the oriented peptide library method described below and in U.S. Pat. No. 5,532,167, the disclosure of which is incorporated by reference herein. See e.g., Rizzuto CD, et. al., Science 280(5371):1949-53 (1998).

TABLE 1
Sequences for Exemplary CXCR4-binding Peptides and Analogs Thereof

| CPI | Peptide Sequence | | | | | | | | | | | | | | | | | SEQ IDs |
|---|---|---|---|---|---|---|---|---|---|---|---|---|---|---|---|---|---|---|
| 1221 | | | M | A | R | S | L | I | W | R | P | A | K | A | K | K | K | SEQ ID NO:1 |
| 1312 | | | | | | | | | | | | | | | | | A | SEQ ID NO:2 |
| 1310 | | | | | | | | | | | | | | | | A | | SEQ ID NO:3 |
| 1301 | | | | | | | | | | | | | | | A | | | SEQ ID NO:4 |
| 1306 | | | | | | | | | | | | | A | | | | | SEQ ID NO:5 |
| 1295 | | | | | | | | | | | A | | | | | | | SEQ ID NO:6 |
| 1305 | | | | | | | | | | A | | | | | | | | SEQ ID NO:7 |
| 1304 | | | | | | | | | A | | | | | | | | | SEQ ID NO:8 |
| 1308 | | | | | | | | A | | | | | | | | | | SEQ ID NO:9 |
| 1307 | | | | | | | A | | | | | | | | | | | SEQ ID NO:10 |
| 1328 | | | | | | A | | | | | | | | | | | | SEQ ID NO:11 |
| | | | | | A | | | | | | | | | | | | | SEQ ID NO:12 |
| 1251 | | Ac | M | A | R | S | L | I | W | R | P | A | K | A | K | K | K | SEQ ID NO:13 |
| 1334 | | | | | | | | | | G | P | | | | | | | SEQ ID NO:14 |
| 1302 | | | | | | | | | | | Tic | | | | | | | SEQ ID NO:15 |
| 1303 | | | | | | | | | | | | Aib | | | | | | SEQ ID NO:16 |
| 1293 | | | | | | | | | w | | | | | | | | | |
| 1309 | | | | | | | | | 2-NaI | | | | | | | | | SEQ ID NO:17 |
| 1291 | | | | | | | | | 1-NaI | | | | | | | | | SEQ ID NO:18 |
| 1296 | | | | | | | F | | | | | | | | | | | SEQ ID NO:19 |
| 1331 | K | K | K | A | R | S | L | I | W | R | P | A | K | A | K | K | K | SEQ ID NO:20 |
| 1300 | F | H | E | F | R | S | L | I | W | R | P | A | K | A | K | K | K | SEQ ID NO:21 |
| 1299 | Y | H | E | F | R | S | L | I | W | R | P | A | K | A | K | K | K | SEQ ID NO:22 |
| 1247 | | | | | R | S | L | I | W | R | P | A | K | A | K | K | K | SEQ ID NO:23 |
| 1248 | | | | | I | S | L | I | W | R | P | A | K | A | K | K | K | SEQ ID NO:24 |
| 1250 | | | | | R | S | L | I | W | R | P | A | K | | | | | SEQ ID NO:25 |
| 1249 | | | M | A | R | S | L | I | W | R | P | A | K | K | K | | | SEQ ID NO:26 |
| 1289 | | | M | A | R | S | L | I | W | R | P | A | K | A | R | R | R | SEQ ID NO:27 |
| 1297 | | | | A | R | S | L | I | W | R | P | A | R | R | R | R | R | SEQ ID NO:28 |
| 1365 | | | | A | R | S | L | I | 2-NaI | R | A | A | R | R | 2-NaI | R | R | SEQ ID NO:29 |
| 1366 | | | Ac | A | R | S | L | I | 2-NaI | R | A | A | R | R | 2-NaI | R | R | SEQ ID NO:30 |
| 1330 | | | F | A | R | S | L | I | E | R | A | A | R | R | W | R | R | SEQ ID NO:31 |
| 1372 | | | M | A | R | S | L | I | W | E | P | A | R | R | W | R | R | SEQ ID NO:32 |
| 1373 | | | M | A | R | S | L | I | W | R | P | A | E | R | W | R | R | SEQ ID NO:33 |
| 1374 | | | M | A | E | S | L | I | W | R | P | A | R | R | W | R | R | SEQ ID NO:34 |
| 1377 | | | M | A | R | S | L | I | W | R | P | A | R | E | W | R | R | SEQ ID NO:35 |
| 1381 | | | | A | R | S | L | I | 2-NaI | R | L | A | R | R | 2-NaI | R | R | SEQ ID NO:36 |
| 1382 | | | | A | R | S | | I | W | R | L | A | R | R | W | R | R | SEQ ID NO:37 |
| 1384 | | | | A | R | S | L | I | Cl—F | R | L | A | R | R | Cl—F | R | R | SEQ ID NO:38 |
| 1389 | | | F | A | R | S | L | I | 2-NaI | E | A | A | R | R | 2-NaI | R | R | SEQ ID NO:39 |
| 1390 | | | F | A | R | S | L | I | 2-NaI | A | | A | R | R | 2-NaI | R | R | SEQ ID NO:40 |
| 1379 | | | Ac | a | r | s | I | I | 2-NaI | r | a | a | r | r | 2-NaI | r | r | |
| 1410 | | | F | A | R | S | L | I | 2-NaI | R | L | A | R | R | 2-NaI | R | R | SEQ ID NO:41 |
| 1411 | | | Y | A | R | S | L | I | 2-NaI | R | L | A | R | R | 2-NaI | R | R | SEQ ID NO:42 |
| 1457 | | | | F | R | S | L | I | 2-NaI | R | L | A | R | R | 2-NaI | R | R | SEQ ID NO:43 |
| 1456 | | R | R | A | R | S | L | I | 2-NaI | R | A | A | R | R | 2-NaI | R | R | SEQ ID NO:44 |
| 1458 | | | | A | R | S | L | I | 2-NaI | R | Tic | A | R | R | 2-NaI | R | R | SEQ ID NO:45 |
| 1448 | | | Ac | A | R | S | L | I | 2-NaI | R | A | A | R | R | 2-NaI | R | R | SEQ ID NO:46 |
| 1443 | | | | A | R | S | L | I | 2-NaI | R | H | A | R | R | 2-NaI | R | R | SEQ ID NO:47 |
| 1424 | K | K | K | A | R | S | L | I | 2-NaI | R | L | A | R | R | 2-NaI | R | R | SEQ ID NO:48 |
| 1425 | | | | A | R | S | L | I | W | R | L | A | R | R | W | R | R | SEQ ID NO:49 |
| 1426 | | | | r | r | 2-naI | r | r | a | I | r | 2-naI | I | I | s | r | a | |
| 1292 | | | | A | R | S | L | I | W | R | P | A | K | A | K | K | K | SEQ ID NO:50 |
| 1298 | | | M | A | R | S | T | I | W | R | P | A | K | A | K | K | K | SEQ ID NO:51 |
| 1329 | | | M | A | A | S | L | I | W | R | P | A | K | A | K | K | K | SEQ ID NO:52 |
| 1332 | | | M | A | R | S | L | I | W | R | P | A | R | R | R | R | R | SEQ ID NO:53 |
| 1629 | | | | A | R | S | L | I | F4F | R | L | A | R | R | 2-NaI | R | R | SEQ ID NO:54 |
| 1630 | | | | A | R | H | L | I | 2-NaI | R | H | A | R | R | 2-NaI | R | R | SEQ ID NO:55 |
| 1631 | | | | H | R | S | L | I | 2-NaI | R | H | A | R | R | 2-NaI | R | R | SEQ ID NO:56 |
| 1632 | | | | | | | | | 2-NaI | R | H | A | R | R | 2-NaI | R | R | SEQ ID NO:57 |
| 1633 | | | | A | R | S | L | I | 2-NaI | R | L | A | R | R | F4F | R | R | SEQ ID NO:58 |
| 1634 | | | | A | R | S | L | I | 2-NaI | R | tic | A | R | R | 2-NaI | R | R | |
| 1641 | | | | | | | | 2-NaI | 2-NaI | R | H | A | R | R | 2-NaI | R | R | SEQ ID NO:59 |
| 1701 | | | | A | R | S | L | I | 2-NaI | R | P | A | R | R | 2-NaI | R | R | SEQ ID NO:60 |

Certain embodiments of the invention are described in the following examples, which are not meant to be limiting.

### EXAMPLES

### Example 1

### Preparation of Tagged CXCR4 and Screening of Peptide Libraries

Using standard techniques known by those skilled in the art, various CXCR4 vectors were prepared for the baculovirus expression system containing epitope tags that allowed for easier purification of the receptor. Tags may be incorporated at the N- or C-terminus of proteins. For certain preparations of CXCR4, tags were incorporated at the C-terminus of the receptor to determine the receptor's ligand-binding properties at the N-terminal region of the molecule to allow easier purification of the receptor. In certain others, tags were placed at the N-terminus of proteins. For CXCR4, tags may be incorporated either at the N- or C-termini of the receptor.

There were no commercially available baculovirus transfer vectors with C-terminal tags. The construction of C terminal 6×His tagged and C-terminal FLAG constructs are provided below as examples. Alternative tags may include, for example, GST, V5, Xpress, c-myc, HA, CBD, and MBP. These constructs were made using standard techniques known by those skilled in the art.

The 6×His tag enables a one-step purification using nickel chelation. The cDNA for CXCR4 was isolated from a spleen cDNA library using Polymerase Chain Reaction (“PCR”) and primers for the 3′ and 5′ ends of CXCR4, as well as to the middle of the gene. To create a C-terminal 6×His tag, CXCR4 was subcloned into an E. coli vector, pET30a, with a C-terminal 6×His tag. The newly created CXCR4-6×His was then excised and ligated into pBlueBac, a baculovirus transfer vector (Invitrogen, Carlsbad, Calif.). The construct was analyzed using both restriction digest and sequencing, and transfected into Sf9 insect cells (Pharmingen, San Diego, Calif.) for expression as typically done by those skilled in the art of protein expression.

A C-terminal bacterial FLAG construct was available from Sigma (St. Louis, Mo.) (pFLAG-CTC). A similar strategy using standard techniques was employed for the construction of this vector. The CXCR4 was subcloned into the pFLAG-CTC plasmid, excised with the C-terminal FLAG tag and then ligated into the digested pBlueBac vector. The construct was analyzed using both restriction digest and sequencing, and transfected into Sf9 or High Five insect cells for expression.

To express the CXCR4 gene in Sf9 or High Five cells, the pBlueBac vector containing the CXCR4 insert was cotransfected with Bac-N-Blue DNA using cationic liposome mediated transfection using standard techniques. The CXCR4 was inserted into the baculovirus genome by homologous recombination. Cells were monitored from 24 hours posttransfection to 4-5 days. After about 72 hours, the transfection supernatant was assayed for recombinant plaques using a standard plaque assay. Cells which have the recombinant virus produce blue plaques when grown in the presence of X-gal (5-bromo-4-chloro-3-indoyl-β-D-galactoside). These plaques were purified and the isolate was verified by PCR for correctness of recombination using standard techniques. From this, a high-titer stock was generated and infection performed from this stock for expression work using standard techniques. Controls for transfection include cells only and transfer vector.

Sf9 or High Five cells were maintained both as adherent and suspension cultures using standard techniques known to those skilled in the art. The adherent cells were grown to confluence and passaged using the sloughing technique at a ratio of 1:5. Suspension cells were maintained in spinner flasks with 0.1% pluronic F-68 (to minimize shearing) for 2-3 months by sub-culturing to a density of 1×10⁶ cells/ml.

A time course after infection with recombinant virus was used to define optimal growth conditions for expression using standard techniques. Aliquots of cells from spinner flasks were taken for this time course, centrifuged at 800×g for 10 minutes at 4° C. and both supernatant and pellet assayed by SDS-PAGE/Western blot analysis. FIG. 2a shows the SDS-PAGE/Western Blots of cell lysate containing CXCR4 and purified CXCR4 stained with coomassie. The CXCR4 was expected to be in the membrane fraction (pellet). All viable systems were assayed in this fashion for levels of expression. The systems with the best expression levels was assayed for activity using a standard binding assay on a membrane preparation using SDF-1 (Chemicon, Temecula, Calif.) and [¹²⁵I]-SDF-1 (New England Nuclear, “NEN”, Boston, Mass.).

The membrane fraction was isolated by first pelleting the whole Sf9 cells (800×g for 10 minutes at 4° C.), then resuspending the pellet in a lysis buffer with homogenization. Typical lysis buffer is around neutral pH and contains a cocktail of protease inhibitors, all of which are standard techniques for those skilled in the art. Membranes were pelleted.

Solubilization was conducted using varying NaCl concentrations. Despite conventional thinking, the step of solubilization using low salt, for example, low calcium and magnesium concentrations substantially in the absence of NaCl provided unexpected optimal conditions for solubilization when compared for quantity and activity. Having 0.0 nM NaCl, although counter-intuitive, provided the best conditions when solubilizing and/or immobilizing candidates with the binding property of CXCR4. The solubilization of the receptor by different detergents (such as, but not limited to, β-dodecylmaltoside, n-octyl-glucoside, CHAPS, deoxycholate, NP-40, Triton X-100, Tween-20, digitonin, Zwittergents, CYMAL, lauroylsarcosine, etc.) was compared for quantity and activity. A candidate for isolation was carried through for purification as described below.

After determining an appropriate detergent for solubilization and activity, such as, for example, Np-40, CXCR4 was purified from the membrane fraction. The exact purification scheme will depend on the construct chosen, which is subject to activity and ease of solubilization. The skilled artisan can readily construct a purification scheme using only routine experimentation. For purification of the 6×His-tagged CXCR4, the membrane fraction was loaded onto a Ni-NTA column (Qiagen, Valencia, Calif.) in the presence of detergent, washed extensively, and eluted with imidazole. Purification of the FLAG-tagged CXCR4 was performed using the anti-FLAG M2 affinity matrix (Sigma, St. Louis, Mo.) in the presence of NP-40 and eluted with glycine. The purification was performed in the presence of NP-40 in the experiment described above. Activity of the purified receptor was assessed using a standard binding/displacement assay using SDF-1 and [¹²⁵I]-SDF-1.

Peptides for libraries were assembled on Rink amide resin (NovaBiochem (San Diego, Calif.), substitution level 0/0.54 mmol/g) using an Applied Biosystems 433A synthesizer (Foster City, Calif.) via 9-fluorenylmethyloxycarbonyl/tert.-butyl (“Fmoc”/“tBu”) based methods. tBu was used for the protection of side-chains of Asp, Glu, Ser, Thr, and Tyr, tert.-butyloxycarbonyl (“Boc”) for Lys and Trp, 2,2,4,6,7-pentamethyldihydrobenzofuran-5-sulfonyl (“Pbf”) for Arg, and triphenylmethyl (“trityl”, “Trt”) for Cys, His, Asn and Gln. The scale of the synthesis was 0.20 mmol. The resin was initially washed with N-methylpyrrolidinone (“NMP”) followed by a 1×3 minutes and 1×7.6 minutes treatment of piperidine:NMP (1:4) for N^{α}-Fmoc removal. All Fmoc-amino acids were coupled with N-[(1H-benzotriazol-1-yl)(dimethylamino)methylene]-N-methylmethanaminium hexafluorophosphate N-oxide (“HBTU”) according to the manufacturer's protocol: (a) 1.0 mmol of derivatized amino acid was dissolved in 2.1 g of NMP; (b) 0.9 mmol of 0.5 M HBTU in N,N-dimethylformamide (“DMF”) was added to the amino acid cartridge and the solution was mixed for 6 minutes; (c) 1.0 mL of 2.0 M N,N-diisopropylethylamine (“DIEA”) in NMP was added to the cartridge; (d) the HBTU solution was transferred to the resin and reacted for 40 minutes at ambient temperature while mixing. The resin was filtered and rinsed six times with a total of 90 ml of NMP and the cycle was repeated. In the one pot method to construct the highly degenerate oriented peptide libraries, a batch of resin was allowed to react with mixtures of the combinatorial amino acids without any partitioning of the resin.

Adjusting the concentrations of the amino acids in the starting mixture controls the relative coupling rates, thereby ensuring equal incorporation of the amino acids in the library.

The optimization of a mixture of natural Boc and Fmoc protected amino acids for the one pot synthesis has been previously described (see e.g., U.S. Pat. No. 5,225,533; Ivanetich, et. Al., Combinatorial Chemistry, vol 267, Academic Press, San Diego, Calif. USA, p 247-260 (1996); Buettner, et al., Innovations and Perspectives in Solid Phase Synthesis: Peptides, Proteins, and Nucleic Acids, Mayflower Worldwide Ltd., Birmingham, UK, p 169-174 (1994); Ostresh, et al, Biopolymers 34:1681-9 (1994); Songyang, et. al., Methods in Mol Biol 87:87-98 (1998); and Herman, et al., Molecular Diversity 2:147-155 (1996). Cleavage reactions were performed by stirring the peptidyl-resin in trifluoroacetic acid (“TFA”):H₂O:anisole:triisopropylsilane (“iPr₃SiH”) (87.5:5:5:2.5, ˜6 mL) for 3 hours at 25° C. (see e.g., Herman et al., 1996). The filtrates were collected and the resin was further washed with TFA. Cold (−78° C.) diethyl ether was added to the combined extracts and the solution was cooled to −78° C. After removing the supernatant, the obtained precipitate was washed several times with cold ether, dissolved in glacial acetic acid and lyophilized.

For cyclic peptide libraries, Fmoc-Asp(OH)-ODmab (Dmab, 4-[N-(1-(4,4-dimethyl-2,6-dioxoxcyclohexylidene)-3-methylbutyl)amino]-benzyl) was side-chain anchored to Rink amide resin followed by chain elongation as described above. Following linear assembly, removal of the Dmab and Fmoc group was accomplished by treatments with hydrazine:DMF (1:49) for 7 minutes and piperidine:NMP (1:4) for 6×3 minutes, respectively. The resin was transferred to a syringe containing a polypropylene frit for manual cyclization. On-resin head-to-tail cyclization was performed using 7-azabenzotriazol-1-yloxy)-tris(pyrrolidino)phosphonium hexafluorophosphate (“PyAOP”):DIEA (1:2, 4 equiv) in a solution containing 1% Trition X in NMP:DMF:dichloromethane, methylene chloride, DCM) (1:1:1) for 2 hours at 55° C. The unreacted linear precursor was treated with Fmoc-Nva-OH/PyAOP/DIEA (“Nva”, “norvaline”)(1:1:2, 4 equiv) in DMF for 1×18 hours and 1×3 hours. Subsequent cleavage and side-chain deprotection as described above yielded a mixture containing a cyclic peptide library and the corresponding linear (uncyclized) sequences. The desired cyclic peptide library was purified to remove the linear contaminants by reversed-phase high performance liquid chromatography (“RP-HPLC”).

Peptides and peptide libraries were characterized by HPLC, MALDI-TOF MS and Edman degradation. MALDI-TOF MS analysis is capable of detecting the presence of high-molecular weight impurities due to incomplete deprotection, deblocking, or re-alkylation. Edman degradation provides quantitative information about the amount of each amino acid in each degenerate position in a library.

The initial libraries synthesized had single, non-degenerate orienting amino acids (i.e., M-X-X-X-X-R-X-X-X-X-A, where X is a degenerate equimolar mixture of all amino acids except cysteine). Cyclic libraries (head-to-tail) were also prepared with single, non-degenerate orienting amino acids. Through the use of these initial libraries, the optimal residues at some degenerate positions become defined and secondary libraries were made fixing these positions. For example, the head to tail cyclized library cyclo(M-X-X-X-X-R-X-X-X-X-N) indicated that the −4 position (from the fixed R) should be lysine, the −2 position should be aspartate, the −1 position should be histidine, and the +3 position should be lysine so the secondary library was cyclo(M-K-X-D-H-R-X-X-K-N) (SEQ ID NO: 61).

An oriented linear peptide library was applied to a column containing immobilized CXCR4 and a small fraction of isolated high affinity peptides. A schematic diagram showing the peptide library using binding domains can be seen in FIG. 2. After washing, bound peptides were eluted from the column. Next, bound peptides and the entire library applied to the column were submitted individually to Edman degradation, to determine the distribution of amino acids as a function of position. Finally, the preferences of amino acids at the degenerate positions was determined. For example, if serine was 5% of the amino acids at position +1 in starting library but 15% of the amino acids in position +1 in the high affinity peptides, there would be a selection for serine at the +1 position. A preference value of 3 at that position would be obtained. Table 2 provides a selective review of the use of the peptide library method with binding domains.

TABLE 2
Use Of Oriented Linear Peptide Libraries To Determine Preferred
Amino Acids For Binding Domains
(residue used for orienting sequence is shown with underline)
(p = phospho-) -- “pX”

| Binding Domain | Preferred Peptide | Kd (nM) | |
|---|---|---|---|
| PDZ | KKKKETDV | 42 | SEQ ID NO:62 |
| Src | EPQpYEEIPIYLK | 80 | SEQ ID NO:63 |
| 14-3-3 | RLSHpSLP | 55.7 | SEQ ID NO:64 |
| SH2 (src) | pYEEIY | 100 | SEQ ID NO:65 |
| SH3 (amphiphysin) | PXRPXR | | SEQ ID NO:66 |
| SHC | NPXpY | | |
| Lim | GPHydGPHydY/F | | SEQ ID NO:67 |

### Example 2

### Preparation and Screening of GST Tagged CXCR4

Libraries were synthesized on an ABI 433A (Applied Biosystems, Foster City, Calif.) with 9-fluorenylmethoxycarbonyl (Fmoc) protecting groups using a Rink Amide MBHA resin (substitution: 0.54 mmol/gm). To obtain approximately equal coupling of amino acids for degenerate positions, the amounts of amino acids are adjusted empirically after considering literature values. See e.g., Ostresh J M, et. al., Biopolymers 34(12):1681-9 (1994). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS and by amino acid sequencing.

The initial library used a single, non-degenerate basic amino acid (i.e., M-A-X-X-X-X-R-X-X-X-X-K-K-K) (SEQ ID NO: 68). Secondary libraries were made fixing optimal residues found at some degenerate positions. For example, M-A-X-X-X-X-W-X-X-X-X-A-K-K-K (SEQ ID NO: 69) may indicate that the −4 position should be arginine, −1 should be isoleucine, and +1 should be arginine so the secondary library would be M-A-R-X-X-I-W-R-X-X-X-A-K-K-K (SEQ ID NO: 70).

In the case of the 6×His tagged CXCR4, the receptor was exposed to the library, and separation of free and bound peptides was accomplished by pelleting the membranes by centrifugation. The 6×His-tagged CXCR4 purified receptor was incubated with a peptide library, about 1 μmole of peptide and about 1 mmole of binding sites. After incubation, receptor with bound peptide was separated from unbound peptides by centrifugation (receptor·peptide complex in the pellet, unbound peptide in the supernatant). Nonspecifically bound peptides were removed by exhaustive washing, and resuspension of the pellet in low pH (≦2.5) was used to remove the bound peptide. This peptide was sequenced to determine the consensus sequence.

When a FLAG-tagged CXCR4 was used for peptide library work, the receptor was immobilized on an anti-FLAG M2 affinity matrix (St. Louis, Mo.). An additional purification approach used the CXCR4-GST construct and immobilized glutathione (Pierce, Rockford, Ill.).

Both the bound peptide mixture and the starting peptide library were sequenced using standard techniques. The amounts of each amino acid, as a function of position, were determined. Preference values for each amino acid at each position were calculated by comparing the amounts of amino acids present in the starting library and bound fraction of peptides. These procedures were used to generate preferred sequences of peptides interacting with many binding domains and have been described in Table 2.

Also, secondary libraries were sequenced incorporating information from the initial library. For the first round of characterization, phage display technology is also used to identify preliminary binding motifs. The phage display method provides for the identification of motifs of natural amino acids. Phage display technology involves the insertion of DNA sequences into a gene coding for one of the phage coat proteins. The gene is inserted in a particular location so that the expressed protein insert can interact with other molecules. As a result, the encoded peptide or protein sequence will be presented on the surface of the phage and exposed for binding. By inserting degenerate nucleotides, each phage can express a different peptide sequence (“a phage library”). Incubation of this phage library with the immobilized receptor can be used to identify sequences which specifically bind to the receptor. Even weak signals can detected because they can be amplified by growing the isolated phage. Information derived from phage display is applicable to affinity purification methods using synthetic libraries containing novel amino acid analogs or cyclic peptides to select ligands that have enhanced pharmaceutical characteristics. The use of initial, secondary and tertiary libraries provided a complete definition of the specificity of the binding site.

Once preferred amino acids residues were identified using high preference values by CXCR4 at the degenerate positions of the library, specific peptides were synthesized by methods as employed for library synthesis. Peptides were then purified by HPLC and compositions confirmed by MALDI-TOF MS.

Relative affinities were measured by modifying the radiolabel binding assay used in receptor purification. Therefore, the ability of these peptides to displace [1²⁵1]-SDF-1 from purified CXCR4 membranes was measured.

### Example 3

### Preparation, Screening and Analysis of Tagged CXCR4 Binding Compounds

Cloning and Expression

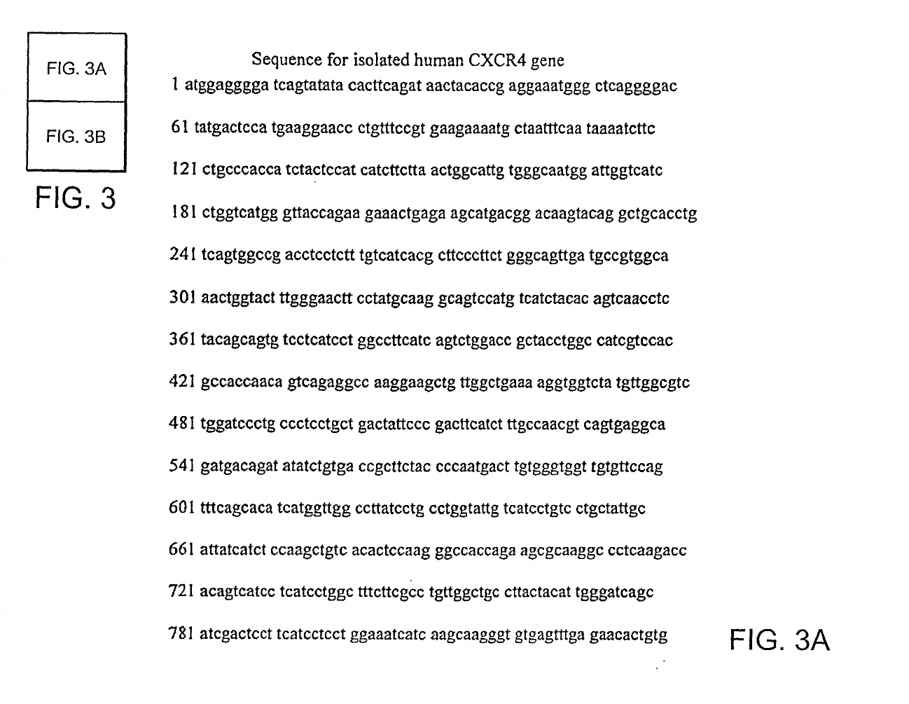

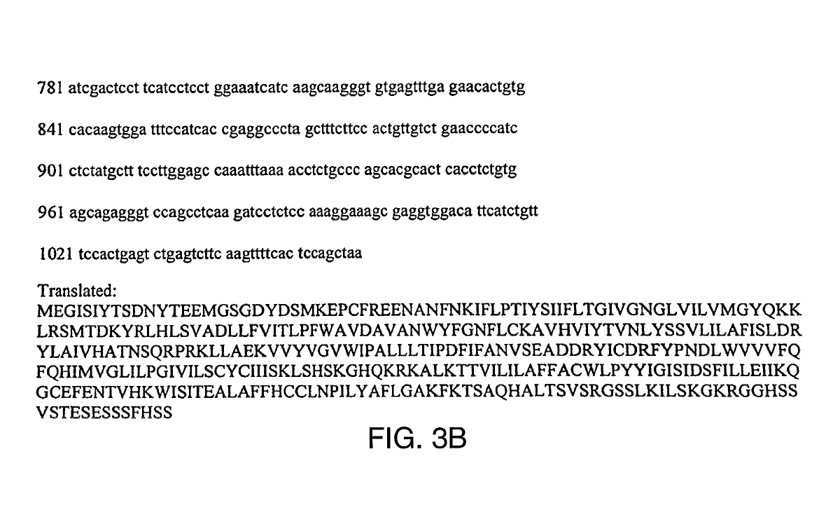

CXCR4 was isolated from a spleen cDNA library in two halves and spliced together. These two fragments were isolated using PCR technology and primers to the 3′ and 5′ ends and the middle of the CXCR4 gene. A full-length clone was not isolated with the 3′ and 5′ primers; however, two halves were isolated and ligated together using a unique BamH I site in the gene. The identity of the construct was confirmed by sequencing. The sequence of the isolate is in FIG. 3. An alternate splice shorter form was also isolated, which is called CXCR4s. Tags were added to the C-terminus of the receptor for use in immobilizing them for affinity purification assays using standard techniques. The following are specific examples from experiments using the tagging method.

Construction of CXCR4 with C-Terminal Histidine Tag (Insect Select Expression System) A previous construct containing the gene for GnRHR (gonadotropin releasing hormone receptor) was used to make the first CXCR4 construct. The gene for GNRHR was spliced out and replaced by the isolated cDNA for CXCR4. This vector was originally the pet30a vector with the 6×His tag at the C-terminus.

Construction of CXCR4 construct with C-terminal FLAG tag: PCR was performed using the primers 5′ BspE1 CXCR4 and 3′ Bgl CXCR4 engineered with unique sites for ligation of CXCR4 in frame with the FLAG tag of pFLAG-CTC (a bacterial expression vector) from Sigma. This construct is called CXCR4-FLAG-CTC. CXCR4-FLAG was then removed by digestion and filled in with Klenow fragment. The fragment containing CXCR4-FLAG was ligated into pBluebac 4.5 that was first digested then blunted with Klenow. This final construct is called CXCR4-FLAG. The construct was confirmed by restriction digestion and sequencing using standard techniques. This construct has been used for expression and has been determined to be expressed sufficiently and in active form for use in affinity purification screening.

Construction of CXCR4 Construct with C-terminal GST tag: The newly constructed CXCR4-FLAG cDNA was removed from the CTC vector and subcloned into another construct, CCR5-GST, in place of the CCR5 (using Bgl and BspE1). This created the vector for CXCR4-GST using one step. The construct was confirmed by restriction digestion and sequencing using standard techniques. This construct has been used for expression and has been determined to be expressed sufficiently and in active form for use in affinity purification screening.

Construction of CXCR4 with N-terminal 6×His tag: This construct was prepared by subcloning the CXCR4 into the commercially available vector, pBluebacHis2b (Invitrogen, Carlsbad, Calif.). The construct was confirmed by restriction digestion and sequencing using standard techniques.

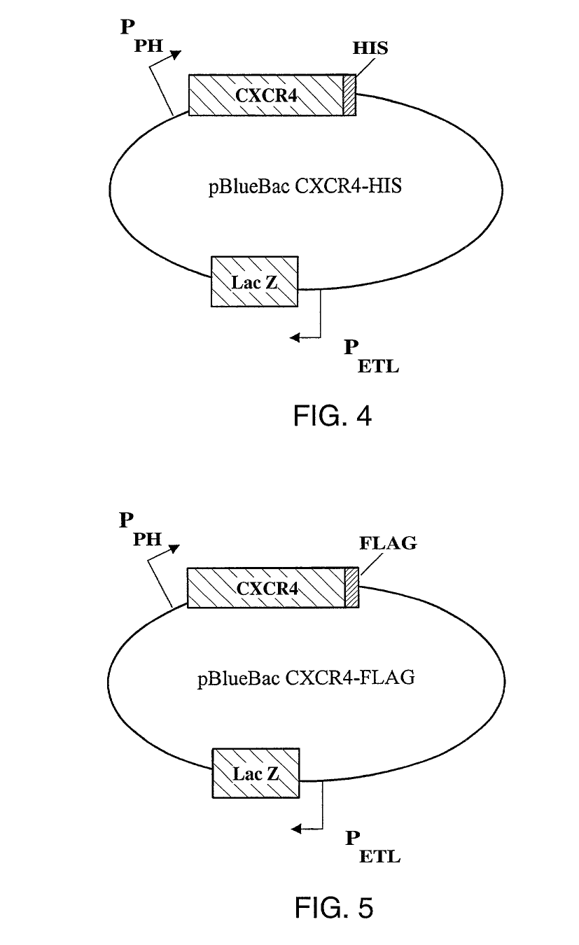

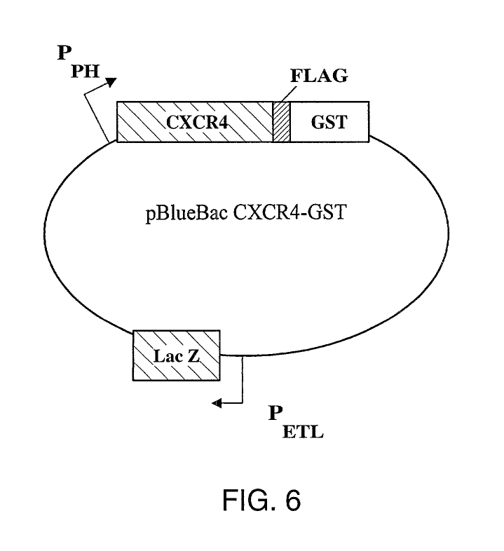

Plasmid maps for these vectors are found in FIGS. 4-6.

The vectors for the three new constructs (for CXCR4-FLAG, CXCR4-GST, and CXCR4-HIS) were used to co-transfect Sf9 cells for the production of a viral stock of each. These viral stocks were purified using a standard plaque assay and then used in experiments to infect for the optimization of expression of CXCR4 with its various C-terminal tags. High Five cells (Invitrogen, Carlsbad, Calif.) were also transfected with these CXCR4 tagged constructs and tested for expression of CXCR4. All constructs were determined to express the appropriately tagged receptor. Expression levels after 72 hours were as much as 5 times greater in High Five cells than those for Sf9 cells. All of the above described experiments were done using standard techniques known to those skilled in the art.

A fourth construct for the expression of CXCR4 was made from the starting vector pBlueBac 4.5 (Invitrogen, Carlsbad, Calif.) to remove the thrombin and enterokinase cleavage sites in the previously described vectors. The GST tag was added into the multiple cloning site by using PCR to generate the GST tag, then ligating into the digested vector (SmaI/EcoRI) using standard procedures known to those skilled in the art. Next, the vector was made compatible with the Gateway cloning technology from Lifetech for ease of manipulation. This was done by ligating into the SmaI site the cassette containing the recombination sites required for this technology (from Lifetech, Rockville, Md.). CXCR4 was amplified using PCR with primers to extend the gene to contain the attachment sites for recombination. Then, the PCR product was incorporated into the baculovirus vector using BP clonase (the enzyme required for homologous recombination) to make a vector for baculovirus expression containing CXCR4 with a C-terminal GST tag without the enterokinase or thrombin cleavage sites. This vector was cotransfected into Sf9 cells for preparation of the virus stock necessary for expression. The virus was plaque purified, and a PCR and sequence checked clone was used for expression of CXCR4. A time course with this construct showed that less proteolysis of the protein was observed and less time was necessary to obtain maximal expression of the receptor.

Activity

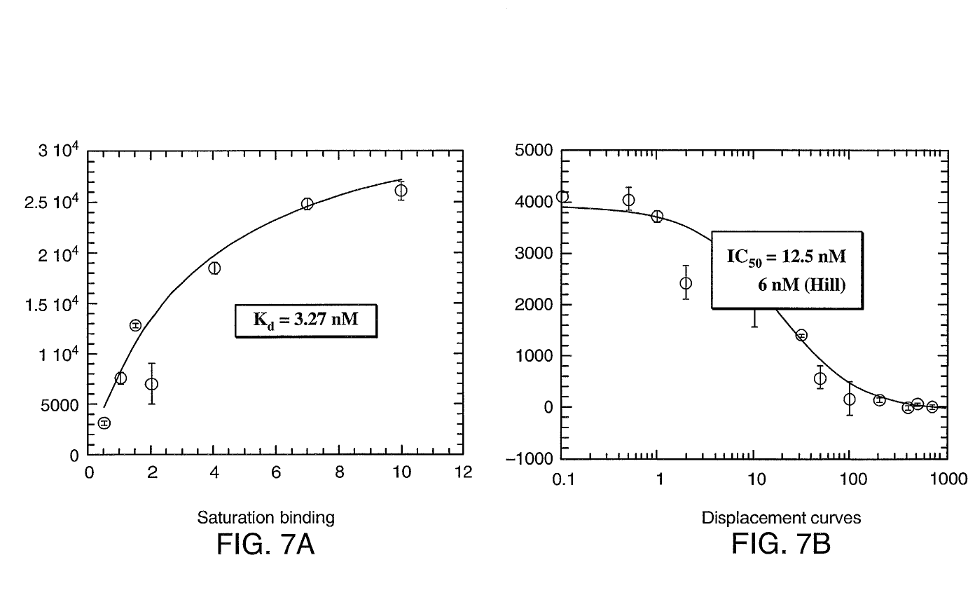

Each of the tagged CXCR4 genes (CXCR4-FLAG, CXCR4-GST, and CXCR4-HIS) were used to co-transfect Sf9 and High Five cells, as described in Example 1. Whole cells from Sf9 and High five cell lines were lysed using hypotonic buffers (10 mM Tris, pH 7.4, 5 mM EDTA), and membrane preparations were made by homogenization and centrifugation using standard techniques known to those skilled in the art. Membrane preparations for CXCR4-GST, CXCR4-FLAG, and CXCR4-HIS were assayed using a standard radioligand binding assay. The radioligand [¹²⁵I]-SDF-1α was incubated with membranes (0.5 μg) in binding buffer at 27° C. for 1 hour with and without unlabelled SDF-1. For filtration and washing, the reaction was transferred to Millipore Multiscreen filter plates (HATF 0.45 μm; pre-blocked with 10% BSA) (Bedford, Mass.), filtered using vacuum, washed 4-5 times with 200 μL ice-cold buffer, and radioactive counts bound were detected using scintillation counting. All points were done in triplicate. Uninfected cells were used as a control for this experiment. To determine the K_{D}, saturation binding was measured using increasing concentrations of [¹²⁵I]-SDF-1α (from 0.5 nM to 10 nM). Nonspecific binding was measured in the presence of 400 nM unlabelled SDF-1α. Competitive binding assays were performed by incubating CXCR4-containing membranes with 0.5 nM [¹²⁵I]-SDF-1α and serial dilutions of unlabelled SDF-1α or peptide ligand. Analyses of K_{D} and IC₅₀ were performed using non-linear curve fitting in Kaleidagraph (software program sold by Synergy Software, Reading, Pa.). FIG. 7 exemplifies radioligand binding studies using membrane preparations of GST-CXCR4 (High Five). Saturation binding demonstrated a K_{D} of 3.27 nM as seen in FIG. 7A. Competitive binding assays yielded an IC₅₀ of 12.5 nM and a Hill coefficient of 0.93 as seen by the displacement curves in FIG. 7B. The assays depicted in FIG. 7 were performed by methods provided herein, and were performed in triplicate. In a preferred embodiment, methods of the invention yield at least approximately 20% active protein.

Solubilization

Both lysed whole cells and membrane preparations have been used for solubilization. Solubilization of the tagged versions of CXCR4 (CXCR4-FLAG, CXCR4-GST, and CXCR4-HIS) have been performed using many different combinations of detergents (NP-40, Triton X-100, β-D-maltoside, n-octylglucoside, CYMAL, Zwittergents, Tween-20, lysophosphatidyl choline, CHAPS, etc.), salts (NaCl, CaCl₂, MgCl₂, MnCl₂, KCl, etc.), buffers (Tris, Hepes, Hepps, Pipes, Mes, Mops, acetate, phosphate, imidazole, etc.), and various pH's (range 6.8-8.2). Conditions for optimal solubilization were found using Zwittergent 3-14 and low salt, e.g. low magnesium and calcium, but no NaCl (0.0 nM NaCl) and buffered at pH 8.1. In a preferred embodiment, at least approximately 20% of the solubilized, immobilized protein is active. In certain highly preferred embodiments, at least approximately 30%, preferably 40%, more preferably 50% and even more preferably 75% of the solubilized, immobilized protein is active.

Immobilization

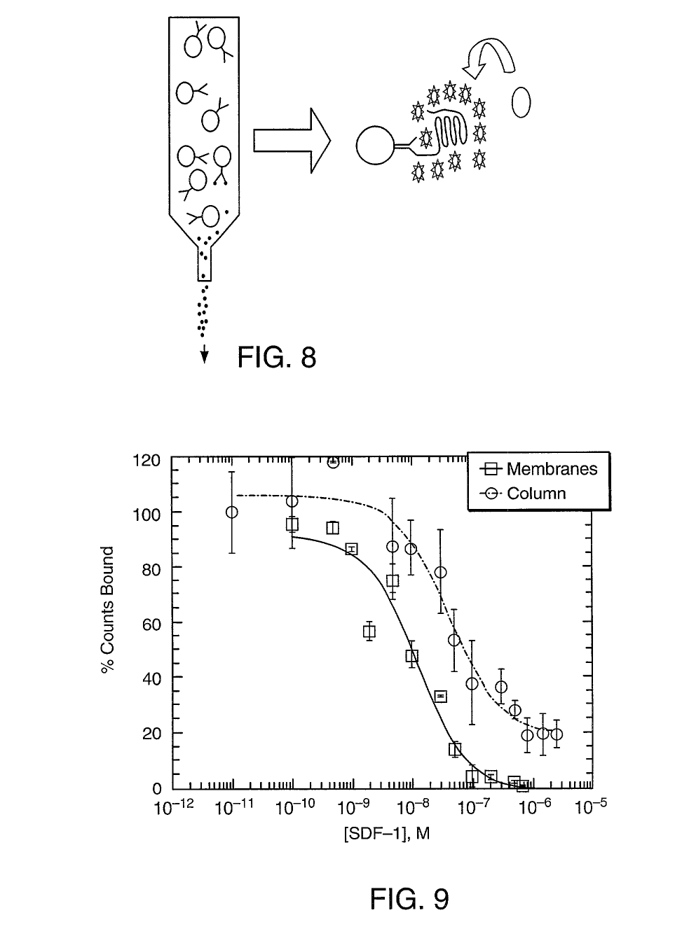

After solubilization, CXCR4-GST were immobilized onto affinity columns for purification and as an active protein ready for use in screening of peptide libraries. A schematic diagram showing the immobilization of GPCRs for affinity purification from libraries is shown in FIG. 8. CXCR4-GST was bound and immobilized onto glutathione-agarose (Pierce, Rockford, Ill.) and glutathione-sepharose (Amersham Pharmacia Biotech, Piscataway, N.J.). The immobilization of the functional protein was accomplished by first solubilizing the receptor in 0.3% NP-40, 10 mM Hepes (or Pipes), pH 7.5, then binding it to the glutathione-sepharose resin in 0.3% NP-40, 10 mM Hepes (or Pipes), pH 7.5, 3 mM CaCl₂, 15 mM MgCl₂. The activity of the immobilized receptor was determined by incubation for 1 hour at 4° C. with the radiolabeled SDF-1α (as with the membrane assay above) and competition with cold SDF-1α. Uninfected cells were used as controls for this activity, as well as the column alone. These experiments demonstrated the ability to immobilize microgram quantities of the receptor in pure form (sufficient for affinity purification screening; see FIG. 8) onto resin in active form. FIG. 9 depicts representative displacement curves for CXCR4. Binding displacement experiments were performed both on CXCR4-containing membranes and on the immobilized receptor with radiolabeled SDF-1 displaced by increasing concentrations of cold SDF-1.

Peptide-Library Synthesis

Libraries were synthesized on an ABI 433A (Applied Biosystems, Foster City, Calif.) with 9-fluorenylmethoxycarbonyl (Fmoc) protecting groups using a Rink Amide MBHA resin (substitution: 0.54 mmol/gm). When a mixture of amino acids was used for degenerate positions, the approximately equal coupling of amino acids was obtained by adjusting the amounts of amino acids empirically after considering literature values. See.e.g., Ivanetich et. al., Combinatorial Chemistry, vol 267, Academic Press, San Diego, Calif. USA, p 247-260 (1996). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS (Louisiana State University) and by amino acid sequencing.

The initial libraries used a single, non-degenerate basic amino acid (i.e., M-X-X-X-X-W-X-X-X-X-A-K-K-K) (SEQ ID NO: 71). Through the use of these initial libraries, the optimal residues at some or all degenerate positions became defined. Secondary libraries were made if not all of the positions were defined, fixing the defined positions.

Screening of Peptide Libraries Using Immobilized CXCR4

With active, large quantities of protein (1 nmol) immobilized to the specific resin (for example, CXCR4-GST to glutathione-sepharose), screening of billions of compounds can take place by incubating them together and allowing the natural preferences and binding affinities to purify the ligands which are preferred by CXCR4. These experiments have been performed with eleven libraries and can be performed with other libraries.

To identify which peptide libraries to screen, a membrane binding assay was developed to use with the peptide libraries. Each peptide library (100 μM final concentration, average molecular weight) was incubated with the receptor (in membranes) and the ability of the peptides in the library to inhibit [¹²⁵I]-SDF1α binding was determined. FIG. 10 is a bar graph depicting the high-throughput ligand-binding inhibition for CXCR4. CXCR4-containing membranes were incubated in the presence of 100 μM library and radiolabeled SDF-1. Percent inhibition was calculated to determine the effect of each library. Peptide libraries with the highest percent inhibition were assayed first.

Approximately 500 mL of 1×10⁶ cell/mL of High Five cells expressing CXCR4-GST were used per affinity purification. Immobilized CXCR4-GST (as described herein) was incubated with 1 mg of the peptide library CPI-10064 (M-A-X-X-X-X-W-X-X-X-X-A-K-K-K) (SEQ ID NO: 69) for 20 minutes at room temperature. Unbound peptides were removed by washing. Bound peptides were eluted with 30% acetic acid. Eluted peptide was filtered using a Centricon-10 (Millipore, Bedford, Mass.) to remove any protein that might have co-eluted with acetic acid. The filtrate was dried under vacuum, dissolved in water and subjected to peptide sequencing.

The sequence for an exemplary consensus motif was identified as M-A-R-S-L-I-W-R-P-A-K-A-K-K-K (CPI-1221) (SEQ ID NO: 1). The affinity for the receptor was determined using standard radioligand displacement methodology. This peptide CPI-1221 was determined to have an IC₅₀ of ˜60 μM.

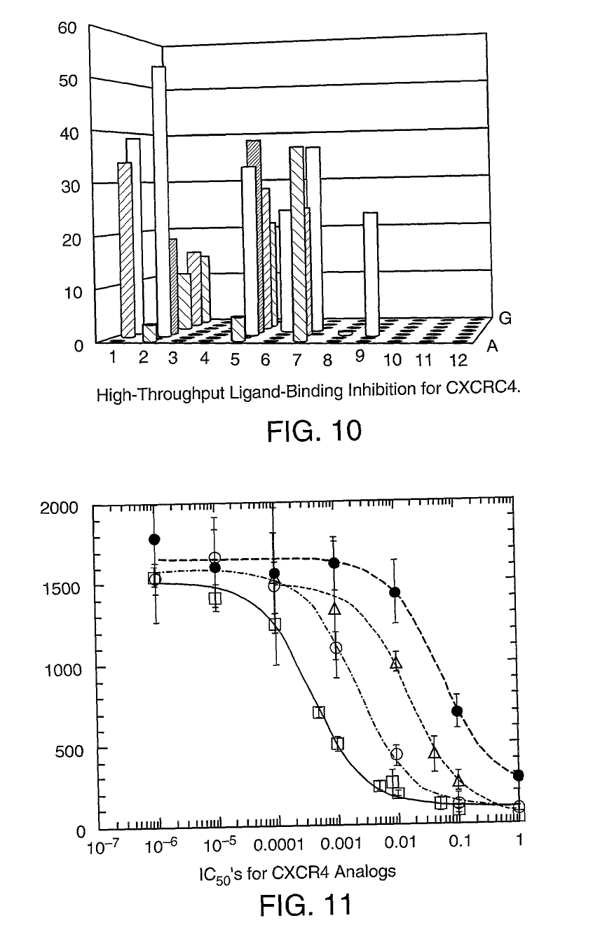

FIG. 11, for example, depicts the IC₅₀ for certain CXCR4 analogs represented by the symbols closed circles (●), open circles (◯), open triangles (Δ) and open squares (□). CPI-1221, CPI-1336, CPI-1289, and CPI-1365 correspond to the symbols closed circles (●), open circles (◯), open triangles (Δ) and open squares (□), respectively. Additionally, certain analogs increased binding inhibition as described herein. CPI-1221, a 15-mer, was the original peptide. Analogs of this peptide were selected having increased specificity as demonstrated by the binding curves in FIG. 11. Various CXCR4 inhibitors were also tested for specificity at other receptors, such as, for example, CXCR2, CCR1, Angiotensin II AT1, and neuropeptide Y. The CXCR4 inhibitors demonstrated no specificity for any of these receptors.

Rational analoging of this peptide was performed as follows. Minimal length of the peptide sequence was determined by deleting from both the N- and C-termini. Approaches to protect against metabolism were conducted using synthetic analogs. Insertion of a rigid segment of the peptide was accomplished with cyclic amino acids such as azetidine-2-carboxylic acid, tetrahydroisoquinoline (Tic), pipecolic acid (Pip), thiazolidine-4-carboxylic acid (Thz), 1-amino-1-cyclopentane-carboxylic acid and 1-amino-1-cyclohexanecarboxylic acid (Sawyer, 1995) as well as α,α-dialkyl residues such as aminoisobutyric acid (Aib) and diethylglycine (Deg). For refinement of activity and bioavailabilty, unnatural amino acids were substituted into a sequence. Analogs of Tyr, Phe (aromatic substitution), Arg, Lys (N^{G}, N^{G}-dimethyl-arginine, 2,3-diaminopropanoic acid), Ala, Gly, Val, Leu (α-allylalanine, 2,3-diphenylglycine, phenylglycine, 4,4,4-trifluorovaline, 5,5,5-trifluoroleucine, β-dimethylaminoleucine) as well as analogs for the other proteogenic amino acids were examined. Hydrophobic unnatural amino acid such as naphthylalanine and cyclohexylalanine substitutions were used successfully. Table 3 is a summary of the substitutions made at the various positions in the peptide CPI-1221 which were synthesized and screened for activity using inhibition of [¹²⁵I]-SDF1α binding and toxicity on Jurkat and CEM cells. In addition, all positions were individually substituted with Ala, several positions were substituted with D-amino acids, the N-terminus of several peptides were acetylated, deletions of the N- and C-termini were made, and several retro-inverso peptides were synthesized.

All of these peptides and analogs were discovered to bind to CXCR4 and inhibit SDF1α binding with varying degrees of activity. Examples of the binding inhibition curves for the original peptide and several analogs can be seen for example in FIG. 11.

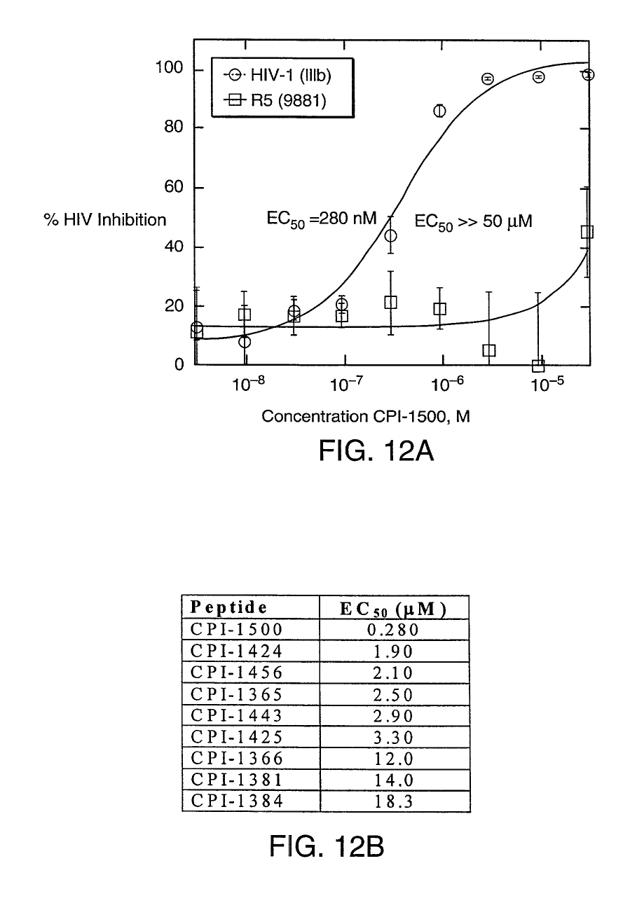

Several of these peptides have also been screened for activity in inhibition of HIV infectivity and determined to be effective in specifically blocking the entrance of HIV into cells with EC₅₀'s in the nM range. For example, the assays depicted in FIG. 12 confirm the inhibition of HIV infection by CXCR4 peptide inhibitors. Specifically, FIG. 12A shows infection of HIV IIIb was inhibited by one such peptide, CPI-1500, with an EC₅₀ of 280 nM. CPI-1500 (ARSLI(2-Nal)R(Tic)ARR(2-Nal)RR) (SEQ ID NO: 72) demonstrated specificity for the HIV IIIb over the R5 virus 9881. FIG. 12B shows that other CXCR4 peptide inhibitors also demonstrated inhibition of HIV IIIb with specificity.

TABLE 3
Peptides and Peptide analogs for CXCR4. Standard one-letter
abbreviations are used for the 20 natural amino acids. Other
abbreviations: 2-Nal (2-naphthalalanine); 1-Nal (1-naphthalalanine);
Cl—F (chloro-phenylalanine); F-4-F (4-phenyl-phenyalanine); Aib
(aminoisobutyric acid); Tic (tetrahydroisoquinoline); hArg(R₂) (homo-
arginine where R = lower alkyl substitution especially ethyl); Hyp
(hydroxyproline); Orn (ornithine); Pya (3-pyridyl-alanine); Phg
(phenylglycine); Dap (2,3-diaminoproprionic acid); and Cha (β-
cyclohexyl-alanine).

A′-B′-C′-D′-E′-E′-F′-C′-G′-F′-C′-B′/C′-F′/C′-C′-C′
Where:

A′ = M, K, hArg(R₂), Orn, Dap
B′ = A, L, I, Cha
C′ = R, K, hArg(R₂), Orn, Dap
D′ = S, A, T
E′ = L, I, V, F, Pya, Phg
F′ = W, 1-Nal, 2-Nal, 4Cl-Phe, 4F-Phe, Pya
G′ = P, H, Tic, A, Hyp, azetidine-2-carboxylic acid

Phage Display

As an alternative or additional method useful in screening for binding compounds and analogs thereof, immobilized functional CXCR4 was used to isolate phage which bind to CXCR4 using standard techniques known to those skilled in the art. Subtraction of the background from the glutathione-sepharose beads, BSA, and SDF1α used in the assay was performed by incubation of the phage library with the mixture of these components. After incubating subtracted phage libraries (i.e., NEB PhD C7C) with the receptor, bound phage were eluted both with the natural CXCR4 ligand (SDF1α) and with glycine, pH 2.2. PhD C7C is a particular phage library with 7 random amino acids between disulfides, and may be obtained from New England Biolabs (“NEB”, Beverly, Mass.). Multiple rounds of screening were performed. Both conditions have provided specific sequences (see Table 4) which bind to CXCR4.

TABLE 4
Phage sequences isolated from CXCR4 screening.
Standard one-letter abbreviations are used for the 20 natural amino acids.

| Phage Sequences | | | | | | | SEQ IDs |
|---|---|---|---|---|---|---|---|
| P | A | H | Y | P | M | L | SEQ ID NO:73 |
| Q | Y | A | T | P | N | K | SEQ ID NO:74 |
| Q | Q | R | S | T | A | F | SEQ ID NO:75 |
| P | F | R | A | T | T | E | SEQ ID NO:76 |
| T | D | K | L | L | L | D | SEQ ID NO:77 |
| H | T | Q | H | V | R | T | SEQ ID NO:78 |
| L | G | V | K | A | P | S | SEQ ID NO:79 |
| D | L | Q | A | R | Y | S | SEQ ID NO:80 |
| S | L | T | E | P | S | L | SEQ ID NO:81 |
| S | T | W | P | L | A | Q | SEQ ID NO:82 |
| R | T | T | S | D | A | L | SEQ ID NO:83 |

### Example 4

### Prevention or Treatment of HIV Infection or AIDS

Presently, certain complications, are encountered during the production, formulation and use of therapeutic peptides, peptidomimetic, or small molecule antagonists or agonists of CXCR4 binding used for the prevention and treatment of AIDS and HIV infection. Biologically appropriate antagonists or agonists that minimize the cost and technical difficulty of commercial production of therapeutic binding compounds of CXCR4 are further contemplated by the present invention. In addition, biologically appropriate antagonists or agonists of CXCR4 binding that do not confer an immunological response to the antagonist or agonist such that it interferes with the effectiveness thereof are contemplated by the invention. Moreover, appropriate formulations that confer commercially reasonable shelf life of the produced antagonist or agonist of CXCR4 binding, without significant loss of biological efficacy are contemplated in the present invention. Furthermore, useful dosages for administration to an individual are contemplated in the present invention appropriate for the prevention and treatment of AIDS and HIV infection.

The identification of appropriate candidates that, alone or admixed with other suitable molecules, that are competent to inhibit CXCR4 binding are contemplated by the invention. Further, the production of commercially significant quantities of the aforementioned identified candidates, which are biologically appropriate for the prevention and/or treatment of AIDS and HIV infection is contemplated. Moreover, the invention provides for the production of therapeutic grade commercially significant quantities of CXCR4 binding antagonists, agonists or derivatives in which any undesirable properties of the initially identified analog, such as in vivo toxicity or a tendency to degrade upon storage, are mitigated.

Methods of preventing and treating AIDS and HIV infection also, after the identification and design of a peptide, peptidomimetic, or small molecule antagonist of CXCR4 binding activity, comprise the step of administering a composition comprising such a compound capable of inhibiting CXCR4 binding as described herein. Administration may be by any compatible route. Thus, as appropriate, administration may include oral or parenteral, including intravenous and intraperitoneal routes of administration. A particularly preferred method is by controlled-release injection of a suitable formulation. In addition, administration may be by periodic injections of a bolus of a composition, or may be made more continuous by intravenous or intraperitoneal administration from a reservoir that is external (e.g., an intravenous bag) or internal (e.g., a bioerodable implant).

Therapeutic compositions contemplated by the present invention may be provided to an individual by any suitable means, directly (e.g., locally, as by injection, implantation or topical administration to a tissue locus) or systemically (e.g., parenterally or orally). Where the composition is to be provided parenterally, such as by intravenous, subcutaneous, intramolecular, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition may comprise part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance.

Useful solutions for parenteral administration may be prepared by any of the methods well known in the pharmaceutical art, described, for example, in REMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. The concept of a controlled release injectable formulation for peptide drugs is well-accepted and offers several advantages. First, for example, bioavailabilities are high. Second, treatment regimens can consist of once per month or per three months (like Abbott's Leupron®), or once per year (e.g. Alza's Viadur®). Third, controlled release injectable formulations substantially reduces the doses that can be used (the Leupron injection dose is 1 mg/day but the 90 day formulation uses is 11.25 mg total). Also, increased efficacy can be achieved if the therapeutic is present continuously to prevent infectivity. This consideration is particularly important in view of the need to approach a cure for this disease by preventing the reformation of slow-to-clear deposits of infection such as the memory T cell compartment. See e.g., Lee, V., ed. Peptide and Protein Drug Delivery. Marcel Dekker, Inc., NY (1991).

Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or cutric acid for vaginal administration.

### Example 5

### Exemplary CXCR4 Binding Compounds

Methods of the invention further contemplate the design of therapeutic agents comprising peptides, peptidomimetics, and/or small molecules which are antagonistic to CXCR4 activity and which are appropriate for the treatment of patients with a disease, such as AIDS. Table 5 further provides both linear and cyclic exemplary peptides that demonstrate enhanced binding affinity and anti-HIV activity, and are resistant to proteolysis. The bolded text represents any changes from the amino acid sequence of CPI-1221, the sequence for which is provided in Table 1. Certain exemplary peptides in Table 5 contain a number in superscript. The superscript represents the position number of the amino acid that corresponds to the respective position in the formula of the general structure of exemplary peptides provided in Table 6. The amino acids corresponding to each of the two position numbers are connected to from a cyclic structure within the peptide. For example, in CPI-2004, the Glutamine at position 8 (based upon the formula provided in Table 6) is connected to the Lysine at position 12 to form a cyclic structure within the peptide. In addition, exemplary peptide sequences are depicted from N-terminal amino acid to the C-terminal amino acid, with the C terminal amino acid having a C-terminal amido group (optionally depicted herein by the # symbol), such as, for example, —NH₂.

TABLE 5

| CPI | Peptide Sequence | |
|---|---|---|
| 1500 | ARSLI(2-Nal)R(Tic)ARR(2-Nal)RR# | SEQ ID NO:72 |
| 1828 | MARSLIWRPAEAKKK# | SEQ ID NO:84 |
| 1831 | MARSLIWRPRKAKKK# | SEQ ID NO:85 |
| 1833 | MARSLIERPAKAKKK# | SEQ ID NO:86 |
| 1837 | MARSLIWRPAKAKQK# | SEQ ID NO:87 |
| 1838 | MARSLIWRPAKALKK# | SEQ ID NO:88 |
| 1841 | MARSLIWRPAKAKEK# | SEQ ID NO:89 |
| 1842 | MARELIWRPAKAKKK# | SEQ ID NO:90 |
| 1843 | MARSLLWRPAKAKKK# | SEQ ID NO:91 |
| 1845 | MARSLIWRPAKLKKK# | SEQ ID NO:92 |
| 1847 | MARSLIWRPAKA(2-Nal)KK# | SEQ ID NO:93 |
| 1848 | (Nle)ARSLIWRPAKAKKK# | SEQ ID NO:94 |
| 1850 | MARSLIQRPAKAKKK# | SEQ ID NO:95 |
| 1853 | MARSLIWRPAKAKKR# | SEQ ID NO:96 |
| 1854 | MARSLIWRPAQAKKK# | SEQ ID NO:97 |
| 1856 | MARSLIWRPAKAWKK# | SEQ ID NO:98 |
| 1870 | Oct-ARSLIWRPAKAKKK# | SEQ ID NO:99 |
| 1956 | MLRSLIWRPAKAKKK# | SEQ ID NO:100 |
| | Cyclo (Glu⁰, Lys⁴) | SEQ ID NO:101 |
| 1960 | EMARKLIWRPAKAKKK# | |
| 1989 | (2Nal)(2Nal)RLARR(2Nal)RR# | SEQ ID NO:102 |
| 1991 | (2Nal)(2Nal)RPARK(2Nal)RR# | SEQ ID NO:103 |
| 1992 | Oct-(2Nal)RPARR(2Nal)RR# | SEQ ID NO:104 |
| 1993 | Cyclo (D-Cys⁸, Cys¹¹) H- | — |
| | (2Nal)(1Nal)cPRCKLAK# | |
| 1994 | (2Nal)(2Nal)RPRAR(2Nal)RR# | SEQ ID NO:105 |
| 1995 | (2Nal)(2Nal)RPARRLRR# | SEQ ID NO:106 |
| 1996 | (2Nal)(2Nal)RPARR(2Nal)RR# | SEQ ID NO:107 |
| 1998 | (2Nal)(1Nal)EPRAKLAK# | SEQ ID NO:108 |
| 2000 | (2Nal)(2Nal)EPARR(2Nal)RR# | SEQ ID NO:109 |
| 2001 | (2Nal)WRPARR(2Nal)RR# | SEQ ID NO:110 |
| 2002 | (2Nal)(2Nal)RPARR(2Nal)RK# | SEQ ID NO:111 |
| 2003 | (2Nal)(2Nal)RPARR(2Nal)AR# | SEQ ID NO:112 |
| 2004 | Cyclo (Glu⁸, Lys¹²) H- | SEQ ID NO:113 |
| | (2Nal)(1Nal)EPRAKLAK# | |
| — | ARSLI(2-Nal)R(Acp)ARR(2-Nal)RR# | SEQ ID NO:114 |
| — | ARSLI(2-Nal)R(Oic)ARR(2-Nal)RR# | SEQ ID NO:115 |
| — | ARSLI(2-Nal)R(Thz)ARR(2-Nal)RR# | SEQ ID NO:116 |
| — | ARSLI(2-Nal)R(N—Me—Phe)ARR(2- | SEQ ID NO:117 |
| | Nal)RR# | |
| — | ARSLI(2-Nal)R(Tpi)ARR(2-Nal)RR# | SEQ ID NO:118 |
| — | ARSLI(2-Nal)RPRAR(2-Nal)RR# | SEQ ID NO:119 |
| — | ARSLI(2-Nal)RLRAR(2-Nal)RR# | SEQ ID NO:120 |
| — | ARSLI(2-Nal)RLARRLRR# | SEQ ID NO:121 |
| — | ARSLILRLARR(2-Nal)RR# | SEQ ID NO:122 |
| — | Cyclo (Glu⁰, Lys⁴) EMARKLI(2- | SEQ ID NO:123 |
| | Nal)R(Tic)ARR(2-Nal)RR# | |
| — | Cyclo (Glu⁸, Lys¹²) ARSLI(2- | SEQ ID NO:124 |
| | Nal)E(Tic)RAK(2-Nal)RR# | |
| — | Cyclo (D-Cys⁸, Cys¹¹) ARSLI(2- | — |
| | Nal)c(Tic)RCR(2-Nal)RR# | |

### Example 6

### More Exemplary Peptides and Peptide Analogs of CXCR4

As already explained above, Table 6 is a summary of the substitutions made at the various positions in certain of the exemplary peptides (cyclic and linear) that are antagonistic to CXCR4 activity. The bolded text represents the peptide sequence identified by CPI-1221 having an amino acid sequence with the appropriate amino acid substitutions based on the formula provided below.

TABLE 6
General Structure: Linear Analogs
X_{N} - X₀ - X₁ - X₂ - X₃ - X₄ - X₅ - X₆ - X₇ - X₈ - X₉ - X₁₀ -
X₁₁ - X₁₂ - X₁₃ - X₁₄ - X₁₅ - X₁₆ - X₁₇
Where:
R₁, R₂, R₃ each independently = H—, alkyl-, aryl-, cycloalkyl
(representing any Rₓ, R_{y} pair, where ‘x’ & ‘y’ represent two
distinct substitutions)
X_{N} = H—, R₁—, R₁C(O)—, R₁C(S)—, R₁C(NR₂R₃)—R₁R₂NC(O)—,
R₁R₂NC(S)—, R₁R₂NC(NR₃R₄)—, R₁OC(O)—
X₀ = Xxxₙ (where n = 0 to 2 amino acids) (e.g., Lys-Lys, Phe-His,
Tyr-His, Arg, Cxx-Cxx)
X₁ = a lipophilic amino acid (e.g., Nle, Met, Leu, Phe), Lys, Arg, Cxx,
Glu, or bond
X₂ = any amino acid, Ala, His, or bond
X₃ = any amino acid, Leu, Ile, Arg, Ala, Glu, Cxx, or bond
X₄ = any amino acid, Ser, Ala, His, Glu, or bond
X₅ = any amino acid, Leu, Thr, Ala, Phe, or bond
X₆ = a lipophilic amino acid (e.g., Leu, Ile), Ala, 2-Nal, or bond
X₇ = a large lipophilic Gln, Glu amino acid (e.g., 1-Nal, 2-Nal, Trp, Ala,
4-F—Phe, 4-Cl—Phe, Acp, Oic, Thz, N—Me—Phe, Tpi), Ala
X₈ = any amino acid, Arg, Gly, Ala, Glu, Cxx, or bond
X₉ = a lipophilic amino acid or imino acid (e.g., Leu, Ala, Tic, Pro), His
X₁₀ = a cationic amino acid (e.g., Arg, Cxx), Ala, Aib, or lipophilic or
aromatic amino acid (e.g., Trp, 2-Nal, et al.)
X₁₁ = any amino acid, Arg, Lys, Cxx, Gln, Ala, Glu
X₁₂ = any amino acid, Arg, Cxx, Leu, Ala, Glu
X₁₃ = a large lipophilic amino acid (e.g., Trp, Leu, 2-Nal), Lys, Arg,
Cxx, Ala, 4-F—Phe, 4-Cl—Phe
X₁₄ = any amino acid, Arg, Lys, Cxx, Gln, Ala, Glu
X₁₅ = a cationic amino acid (e.g., Arg, Lys, Cxx), Ala
X₁₆ = Xxxₘ (where m = 0 to 2 amino acids)
X₁₇ = —OR₁, —NR₁R₂
Cyclic Analogs:
Note: i to i + 4 bridges (lactam in any orientation between Asp or Glu,
and Dab or Lys; disulfides between any pair of Cys or homoCys or Pen)
where ‘i’ relates to the ‘n’ of Xₙ in the Markush structure above & =
1, 2, 4, 5, 6, 8, 9, 10, 11, 12, or 13
Note: D-Xxx¹ to L-Xxxⁱ⁺³ disulfide bridges where Xxx = Cys or Pen,
& i = 1, 2, 3, 5, 6, 8, 9, 10, 11, 12, 13, or 14
Where:
R₁, R₂, R₃ each independently = H—, alkyl-
X_{N} = H—, R₁C(O)—
X₀ = Xxxₙ (where n = 0 to 2 amino acids) (e.g., Lys-Lys, Cxx-Cxx)
X₁ = a lipophilic amino acid (e.g., Nle, Met, Leu, Phe), Lys, Arg, Cxx,
or bond
X₂ = any amino acid (e.g., Ala), or bond
X₃ = any amino acid (e.g., Leu, Ile, Arg, Cxx, Ala), or bond
X₄ = any amino acid (e.g., Ser, Ala, His, Glu) or bond
X₅ = any amino acid, Leu, Thr, Ala, Phe, or bond
X₆ = a lipophilic amino acid (e.g., Leu, Ile), Ala, 2-Nal, or bond
X₇ = a large lipophilic amino acid (e.g., 1-Nal, 2-Nal, Trp, 4-F—Phe,
4-Cl—Phe, Acp, Oic, Thz, N—Me—Phe, Tpi)
X₈ = any amino acid, Arg, Cxx, Gly, or bond
X₉ = a lipophilic amino acid or imino acid (e.g., Leu, Tic, Pro), Ala, His
X₁₀ = a cationic amino acid (e.g., Arg, Cxx), Ala, Aib, or lipophilic or
aromatic amino acid (e.g., Trp, 2-Nal, et al.)
X₁₁ = any amino acid, Arg, Lys, Cxx, Gln
X₁₂ = any amino acid, Arg, Cxx, Leu, Ala
X₁₃ = a large lipophilic amino acid (e.g., Trp, Leu, 2-Nal), Lys, Arg,
Cxx, Ala, 4-F—Phe, 4-Cl—Phe
X₁₄ = any amino acid, Arg, Lys, Cxx, Gln, Ala
X₁₅ = a cationic amino acid (e.g., Arg, Lys, Cxx)
X₁₆ = Xxxₘ (where m = 0 amino acids)
X₁₇ = —NR₁R₂
Standard abbreviations are used for the 20 natural amino acids. Other
abbreviations include, for example:
“Any amino acid” includes all L- or D-amino acids natural or unnatural.

| | | |
|---|---|---|
| Acp | = | 1-aminocyclopropane-1-carboxylic acid |
| Aib | = | 2-amino-isobutyric acid |
| Cxx | = | cationic, amido and urea, linear or cyclic, side chain amino |
| | | acids, as exemplified by —NHC((CH₂)ₙNR₄R₅)CO— (linear |
| | | side chain example), where ‘n’ = 1 to 5; R₄ = H—, alkyl; |
| | | R₅ = H—, alkyl, acyl, —CONHR₆, |
| | | —C(NR₆)NHR₆ (where R₆ = H—, alkyl) |
| 1-Nal | = | L-β-(1-naphthyl)-alanine |
| 2-Nal | = | L-β-(2-naphthyl)-alanine |
| 2-nal | = | D-β-(2-naphthyl)-alanine |
| Oct | = | octanoyl |
| Oic | = | L-octahydroindole-2-carboxylic acid |
| Thz | = | L-thiazolidine-4-carboxylic acid |
| Tpi | = | L-1,2,3,4-tetrahydronorharman-3-carboxylic acid |
| # | = | (C-terminal amido group; e.g., —NH₂) |

### Example 7

### Assay for Inhibition of HIV Infectivity

The anti-HIV activity of exemplary compounds was tested using established adherent HELA cells or Magi cell-based (Magi cell line incorporating the reporter genes) cell line infected with a variety of HIV-1 isolates. Inhibitory activity was determined using a reporter system. The cell line was modified to contain the gene for the HIV-LTR (long terminal repeat) promoter which is used by HIV after infection. This gene was coupled to an enzyme, β-galactosidase, which can cleave a substrate, CPRG (Chlorophenol red β-D-galactoside) which gives rise to a colored signal or FDG (Fluorescein di-Galactoside) which gives rise to a fluorescent signal. Thus, if the cell becomes infected with HIV, it couples to the HIV-LTR promoter, producing the enzyme β-galactosidase, which then cleaves the CPRG substrate giving rise to a measurable absorbance signal, for example, at or about 595 nm. The signal is thus proportional to the amount of infection by HIV, giving a specific measure of how much HIV gets into a cell.

Toxicity was measured with the same cell system by measuring the effect of the inhibitor on cellular metabolism. A standard measure of the toxicity uses the ability of cells to convert the tetrazolium salt MTS (5-(3-carboxymethoxyphenyl)-2-(4,5-dimethylthiazol)-3-(4-sulphophenyl) tetrazolium, inner salt) (Promega, Madison, Wis.) to a colored substance, formazan, giving a direct measurement of the number of viable cells remaining (by measuring the absorbance at or about 450 nm).

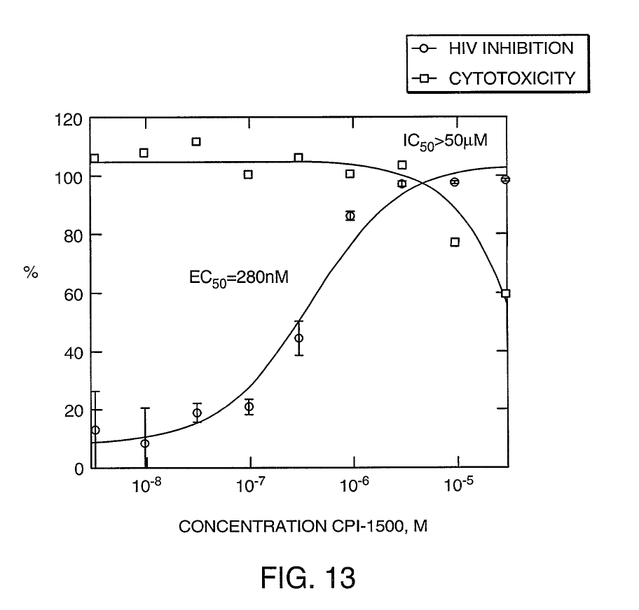

Measurements were taken four days after infection. A well-characterized HIV inhibitor, AZT, was used as a control. EC₅₀'s were determined graphically using Kaleidagraph, a software program sold by Synergy Software, Reading, PA. Representative data for infectivity and viral specificity of CPI-1500 is depicted, for example in FIG. 12A and FIG. 13. Other exemplary compounds which can inhibit HIV infection are also listed in Table 7.

TABLE 7
Inhibition of HIV infection by CXCR4 inhibitors.

| CPI# | Sequence | SEQ ID |
|---|---|---|
| 1500 | ARSLI(2-Nal)R(Tic)ARR(2-Nal)RR | SEQ ID NO:72 |
| 1701 | ARSLI(2-Nal)RPARR(2-Nal)RR | SEQ ID NO:60 |
| 1424 | KKKARSLI(2Nal)RLARR(2-Nal)RR | SEQ ID NO:48 |
| 1456 | RRARSLI(2-Nal)RAARR(2-Nal)RR | SEQ ID NO:44 |
| 1365 | ARSLI(2-Nal)RAARR(2-Nal)RR | SEQ ID NO:29 |
| 1443 | ARSLI(2-Nal)RHARR(2-Nal)RR | SEQ ID NO:47 |
| 1425 | ARSLIWRLARRWRR | SEQ ID NO:49 |
| 1366 | Ac-ARSLI(2-Nal)RAARR(2-Nal)RR | SEQ ID NO:30 |
| 1381 | ARSLI(2-Nal)RLARR(2-Nal)RR | SEQ ID NO:36 |
| 1389 | FARSLI(2-Nal)EAARR(2-Nal)RR | SEQ ID NO:39 |
| 1384 | ARSLI(Cl—F)RLARR(Cl—F)RR | SEQ ID NO:38 |
| 1641 | (2-Nal)-(2-Nal)RHARR(2-Nal)RR | SEQ ID NO:59 |

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of identifying a binding compound for CXC chemokine receptor 4 comprising the steps of:
a) providing a library of two or more molecules;
b) providing a molecule having a binding property corresponding to CXC chemokine receptor 4;
c) binding a molecule from said library of two or more molecules to said molecule having a binding property corresponding to CXC chemokine receptor 4;
d) separating said bound molecule from said library of two or more molecules; and
e) identifying said bound molecule as a binding compound for CXC chemokine receptor 4.

2. The method of claim 1, wherein said library of two or more molecules is selected from the group consisting of linear peptides, cyclic peptides, natural amino acids, unnatural amino acids, peptidomimetic compounds and small molecule compounds.

3. The method of claim 1, wherein said molecule having a binding property corresponding to CXC chemokine receptor 4 is a partially purified CXC chemokine receptor.

4. The method of claim 1, wherein at least one of said two or more molecules is selected from a group consisting of a peptide, a peptidomimetic or small molecule that can substitute for a protein capable of binding to receptors, enzymes or other proteins.

5. The method of claim 1, further comprising the step of solubilizing said molecule having a binding property corresponding to CXC chemokine receptor 4 substantially in the absence of sodium chloride.

6. The method of claim 1, further comprising the step of solubilizing said molecule having a binding property corresponding to CXC chemokine receptor 4 using a buffer having a low salt concentration.

7. The method of claim 1, wherein at least one of said two or more molecules comprises a molecule having an antagonistic effect on CXC chemokine receptor 4 binding activity.

8. The method of claim 1, wherein said library comprises a phage library.

9. The method of claim 1, wherein said steps a, b, c, and d are repeated at least once prior to said step e.

10. The method of claim 1, wherein said molecule having a binding property corresponding to CXC chemokine receptor 4 comprises a CXC chemokine receptor 4 molecule and a tag selected from the group consisting of GST, FLAG, 6×His, C-MYC, MBP, V5, Xpress, CBP, and HA).

11. A binding compound for CXC chemokine receptor 4 identified according to the method of claim 1.

12. A method of preventing HIV infection in a patient, the method comprising administering to said patient a therapeutic composition comprising the compound of claim 1 in a physiological carrier.

13. A method of treating or preventing AIDS in a patient, the method comprising administering to said patient a therapeutic composition comprising the compound of claim 1 in a physiological carrier.

14. A method of treating or preventing AIDS in a patient, the method comprising administering to said patient a therapeutic composition comprising the compound of claim 1 in a controlled release injectable formulation.

15. A computer-aided method for identifying relative binding affinity of a test molecule to CXC chemokine receptor 4, comprising the steps of:
a) entering input data characterizing CXC chemokine receptor 4 into a computer program;
b) entering input data characterizing at least one test peptide-like molecule, each of known sequence but unknown binding affinity;
c) analyzing each applied test peptide-like molecule using the computer program to generate a prediction of a relative binding affinity for each test peptide-like molecule, and outputting such prediction.

16. A method for determining an amino acid sequence motif for an interaction site of a binding compound for CXC chemokine receptor 4, comprising the steps of:
a) contacting a peptide library with a molecule having a binding property corresponding to CXC chemokine receptor 4 under conditions which allow for interaction between said molecule having a binding property corresponding to CXC chemokine receptor 4 and said peptide library;
b) allowing said molecule having a binding property corresponding to CXC chemokine receptor 4 to interact with said peptide library such that a complex is formed between said molecule having a binding property corresponding to CXC chemokine receptor 4 and a subpopulation of library members capable of interacting with said molecule having a binding property corresponding to CXC chemokine receptor 4;
c) separating said subpopulation of library members capable of interacting with said molecule having a binding property corresponding to CXC chemokine receptor 4 from library members that are incapable of interacting with said molecule having a binding property corresponding to CXC chemokine receptor 4;
d) determining a relative abundance of different amino acid residues at each degenerate position within said subpopulation of library members; and
e) determining an amino acid sequence motif for an interaction site of said molecule having a binding property corresponding to CXC chemokine receptor 4, based upon said relative abundance of different amino acid residues at each degenerate position within the library members.

17. An amino acid sequence motif for a binding compound for CXC chemokine receptor 4 identified according to the method of claim 16.

18. An amino acid sequence motif identified according to the method of claim 16 having sequence M-A-R-S-L-1-W-R-P-A-K-A-K-K-K (SEQ ID NO: 1).

19. A binding compound identified according to the method of claim 1 having a sequence selected from the group consisting of P-A-H-Y-P-M-L (SEQ ID NO: 73), Q-Y-A-T-P-N-K (SEQ ID NO: 74), Q-Q-R-S-T-A-F (SEQ ID NO: 75), P-F-R-A-T-T-E (SEQ ID NO: 76), T-D-K-L-L-L-D (SEQ ID NO: 77), H-T-Q-H-V-R-T (SEQ ID NO: 78), L-G-V-K-A-P-S (SEQ ID NO: 79), D-L-Q-A-R-Y-S (SEQ ID NO: 80), S-L-T-E-P-S-L (SEQ ID NO: 81), S-T-W-P-L-A-Q (SEQ ID NO: 82), and R-T-T-S-D-A-L (SEQ ID NO: 83).

20. A binding compound having the amino acid sequence motif for CXC chemokine receptor 4 determined by the method of claim 16.

21. A binding compound identified according to the method of claim 16 having the sequence comprising A′-B′-C′-D′-E′-E′-F′-C′-G′-F′/C′/B′-C′-B′/C′-F′/C′-C′-C′.

22. The method of claim 16, wherein at least one member of said peptide library comprises at least one unnatural amino acid.

23. The method of claim 16, wherein said molecule having a binding property corresponding to CXC chemokine receptor 4 is selected from the group consisting of linear peptides, cyclic peptides, natural amino acids, unnatural amino acids, peptidomimetic compounds and small molecule compounds.

24. The method of claim 16, wherein said peptide library comprises at least one molecule selected from the group consisting of linear peptides, cyclic peptides, natural amino acids, unnatural amino acids, peptidomimetic compounds and small molecule compounds.

25. The method of claim 16, wherein said peptide library is selected from a group consisting of M-X-X-X-X-R-X-X-X-X-A, M-A-X-X-X-X-R-X-X-X-X-K-K-K (SEQ ID NO: 68), M-A-X-X-X-X-W-X-X-X-X-A-K-K-K (SEQ ID NO: 69), M-A-R-X-X-1-W-R-X-X-X-A-K-K-K (SEQ ID NO: 70), M-X-X-X-X-W-X-X-X-X-A-K-K-K (SEQ ID NO: 71), cyclo(M-X-X-X-X-R-X-X-X-X-N), and cyclo(M-K-X-D-H-R-X-X-K-N) (SEQ ID NO: 61).

26. The method of claim 16, wherein said peptide library is selected from a pre-determined CPI peptide sequence.

27. A library comprising members based upon an amino acid sequence motif for an interaction site of a binding compound for CXC chemokine receptor 4, the motif being determined by permitting at least one peptide member from a peptide library to interact with said binding compound for CXC chemokine receptor 4, and determining an amino acid sequence of at least one peptide that interacts with said binding compound for CXC chemokine receptor 4.

28. A method of solubilizing or immobilizing a compound corresponding to the binding property of CXC chemokine receptor 4, wherein the solubilization or immobilization is conducted substantially in the absence of sodium chloride when determining a compound corresponding to the binding of CXC chemokine receptor 4.

29. A method of solubilizing or immobilizing a compound corresponding to the binding property of CXC chemokine receptor 4, wherein the solubilization or immobilization is conducted by a using a low salt concentration when determining a compound corresponding to the binding of CXC chemokine receptor 4.

30. The method of claim 29, wherein said low salt concentration comprises a predetermined amount of magnesium and calcium.

31. A CXC chemokine 4 transfer vector comprising a CXC chemokine receptor 4 molecule and a tag selected from the group consisting of GST, FLAG, 6×His, C-MYC, MBP, V5, Xpress, CBP, and HA.

32. A method of using a three-dimensional structure of CXC chemokine receptor 4 in a drug screening assay comprising:
a) selecting a potential drug by performing rational drug design with the three-dimensional structure, wherein said selecting step is performed in conjunction with computer modeling;
b) contacting the potential drug with a first molecule comprising a first CXC chemokine receptor 4; and
c) detecting the binding of the potential drug with said first molecule; wherein a potential drug is selected as a drug if the potential drug binds to said first molecule.

33. The method of claim 32, wherein said first molecule is labeled.

34. The method of claim 32, wherein said first molecule is bound to a solid support.

35. A binding compound identified according to the method of claim 1 having the sequence comprising ARSLI(2-Nal)R(Tic)ARR(2-Nal)RR (SEQ ID NO: 72).

36. A binding compound identified according to the method of claim 1 having the sequence comprising ARSLI(2-Nal)RPARR(2-Nal)RR (SEQ ID NO: 60).

37. A binding compound identified according to the method of claim 1 having the sequence comprising KKKARSLI(2-Nal)RLARR(2-Nal)RR (SEQ ID NO: 48).

38. A binding compound identified according to the method of claim 1 having the sequence comprising ARSLI(2-Nal)RAARR(2-Nal)RR (SEQ ID NO: 29).

39. A binding compound identified according to the method of claim 1 having the sequence comprising RRARSLI(2-Nal)RAARR(2-Nal)RR (SEQ ID NO: 44).

40. A binding compound identified according to the method of claim 1 having the sequence comprising H-ARSLI(2-Nal)RHARR(2-Nal)RR (SEQ ID NO: 47).

41. A binding compound identified according to the method of claim 1 having the sequence comprising Cyclo (Glu⁰, Lys⁴) EMARKLI(2-Nal)R(Tic)ARR(2-Nal)RR (SEQ ID NO: 123).

42. A binding compound identified according to the method of claim 1 having the sequence comprising Cyclo (Glu⁸, Lys¹²) ARSLI(2-Nal)E(Tic)RAK(2-Nal)RR (SEQ ID NO: 124).

43. A binding compound identified according to the method of claim 1 having the sequence comprising Cyclo (D-Cys⁸, Cys¹¹) ARSLI(2-Nal)c(Tic)RCR(2-Nal)RR.

44. A binding compound identified according to the method of claim 1 having the sequence comprising Cyclo (Glu⁰, Lys⁴) EMARKLIWRPAKAKKK (SEQ ID NO: 101).

45. A method of treating disease in a patient, the method comprising administering to said patient a therapeutic composition comprising the compound of claim 1 in a physiological carrier.


SEQUENCE LISTING (126 sequences)

SEQ ID NO: 1 - protein, 15 aa, Artificial sequence
  CXCR4-binding peptide
MARSLIWRPAKAKKK

SEQ ID NO: 2 - protein, 15 aa, Artificial sequence
  CXCR4 binding peptide
MARSLIWRPAKAKKA

SEQ ID NO: 3 - protein, 15 aa, Artificial sequence
  CXCR4 binding peptide
MARSLIWRPAKAKAK

SEQ ID NO: 4 - protein, 15 aa, Artificial sequence
  CXCR4 binding peptide
MARSLIWRPAKAAKK

SEQ ID NO: 5 - protein, 15 aa, Artificial sequence
  CXCR4 binding peptide
MARSLIWRPAAAKKK

SEQ ID NO: 6 - protein, 15 aa, Artificial sequence
  CXCR4 binding peptide
MARSLIWRAAKAKKK

SEQ ID NO: 7 - protein, 15 aa, Artificial sequence
  CXCR4 binding peptide
MARSLIWAPAKAKKK

SEQ ID NO: 8 - protein, 15 aa, Artificial sequence
  CXCR4 binding peptide
MARSLIARPAKAKKK

SEQ ID NO: 9 - protein, 15 aa, Artificial sequence
  CXCR4 binding peptide
MARSLAWRPAKAKKK

SEQ ID NO: 10 - protein, 15 aa, Artificial sequence
  CXCR4 binding peptide
MARSAIWRPAKAKKK

SEQ ID NO: 11 - protein, 15 aa, Artificial sequence
  CXCR4 binding peptide
MARALIWRPAKAKKK

SEQ ID NO: 12 - protein, 15 aa, Artificial sequence
  CXCR4 binding peptide
MAASLIWRPAKAKKK

SEQ ID NO: 13 - protein, 15 aa, Artificial sequence
  CXCR4 binding peptide
MARSLIWRPAKAKKK

SEQ ID NO: 14 - protein, 15 aa, Artificial sequence
  CXCR4 binding peptide
MARSLIWGPAKAKKK

SEQ ID NO: 15 - protein, 15 aa, Artificial sequence
  CXCR4 binding peptide
MARSLIWRXAKAKKK

SEQ ID NO: 16 - protein, 15 aa, Artificial sequence
  CXCR4 binding peptide
MARSLIWRPXKAKKK

SEQ ID NO: 17 - protein, 15 aa, Artificial sequence
  CXCR4 binding peptide
MARSLIXRPAKAKKK

SEQ ID NO: 18 - protein, 15 aa, Artificial sequence
  CXCR4 binding peptide
MARSLIXRPAKAKKK

SEQ ID NO: 19 - protein, 15 aa, Artificial sequence
  CXCR4 binding peptide
MARSFIWRPAKAKKK

SEQ ID NO: 20 - protein, 17 aa, Artificial sequence
  CXCR4 binding peptide
KKKARSLIWRPAKAKKK

SEQ ID NO: 21 - protein, 17 aa, Artificial sequence
  CXCR4 binding peptide
FHEFRSLIWRPAKAKKK

SEQ ID NO: 22 - protein, 17 aa, Artificial sequence
  CXCR4 binding peptide
YHEFRSLIWRPAKAKKK

SEQ ID NO: 23 - protein, 13 aa, Artificial sequence
  CXCR4 binding peptide
RSLIWRPAKAKKK

SEQ ID NO: 24 - protein, 13 aa, Artificial sequence
  CXCR4 binding peptide
ISLIWRPAKAKKK

SEQ ID NO: 25 - protein, 9 aa, Artificial sequence
  CXCR4 binding peptide
RSLIWRPAK

SEQ ID NO: 26 - protein, 13 aa, Artificial sequence
  CXCR4 binding peptide
MARSLIWRPAKKK

SEQ ID NO: 27 - protein, 15 aa, Artificial sequence
  CXCR4 binding peptide
MARSLIWRPAKARRR

SEQ ID NO: 28 - protein, 14 aa, Artificial sequence
  CXCR4 binding peptide
ARSLIWRPARRRRR

SEQ ID NO: 29 - protein, 14 aa, Artificial sequence
  CXCR4 binding peptide
ARSLIXRAARRXRR

SEQ ID NO: 30 - protein, 14 aa, Artificial sequence
  CXCR4 binding peptide
ARSLIXRAARRXRR

SEQ ID NO: 31 - protein, 15 aa, Artificial sequence
  CXCR4 binding peptide
FARSLIERAARRWRR

SEQ ID NO: 32 - protein, 15 aa, Artificial sequence
  CXCR4 binding peptide
MARSLIWEPARRWRR

SEQ ID NO: 33 - protein, 15 aa, Artificial sequence
  CXCR4 binding peptide
MARSLIWRPAERWRR

SEQ ID NO: 34 - protein, 15 aa, Artificial sequence
  CXCR4 binding peptide
MAESLIWRPARRWRR

SEQ ID NO: 35 - protein, 15 aa, Artificial sequence
  CXCR4 binding peptide
MARSLIWRPAREWRR

SEQ ID NO: 36 - protein, 14 aa, Artificial sequence
  CXCR4 binding peptide
ARSLIXRLARRXRR

SEQ ID NO: 37 - protein, 13 aa, Artificial sequence
  CXCR4 binding peptide
ARSIWRLARRWRR

SEQ ID NO: 38 - protein, 14 aa, Artificial sequence
  CXCR4 binding peptide
ARSLIXRLARRXRR

SEQ ID NO: 39 - protein, 15 aa, Artificial sequence
  CXCR4 binding peptide
FARSLIXEAARRXRR

SEQ ID NO: 40 - protein, 14 aa, Artificial sequence
  CXCR4 binding peptide
FARSLIXAARRXRR

SEQ ID NO: 41 - protein, 15 aa, Artificial sequence
  CXCR4 binding peptide
FARSLIXRLARRXRR

SEQ ID NO: 42 - protein, 15 aa, Artificial sequence
  CXCR4 binding peptide
YARSLIXRLARRXRR

SEQ ID NO: 43 - protein, 14 aa, Artificial sequence
  CXCR4 binding peptide
FRSLIXRLARRXRR

SEQ ID NO: 44 - protein, 16 aa, Artificial sequence
  CXCR4 binding peptide
RRARSLIXRAARRXRR

SEQ ID NO: 45 - protein, 14 aa, Artificial sequence
  CXCR4 binding peptide
ARSLIXRXARRXRR

SEQ ID NO: 46 - protein, 14 aa, Artificial sequence
  CXCR4 binding peptide
ARSLIXRAARRXRR

SEQ ID NO: 47 - protein, 14 aa, Artificial sequence
  CXCR4 binding peptide
ARSLIXRHARRXRR

SEQ ID NO: 48 - protein, 17 aa, Artificial sequence
  CXCR4 binding peptide
KKKARSLIXRLARRXRR

SEQ ID NO: 49 - protein, 14 aa, Artificial sequence
  CXCR4 binding peptide
ARSLIWRLARRWRR

SEQ ID NO: 50 - protein, 14 aa, Artificial sequence
  CXCR4 binding peptide
ARSLIWRPAKAKKK

SEQ ID NO: 51 - protein, 15 aa, Artificial sequence
  CXCR4 binding peptide
MARSTIWRPAKAKKK

SEQ ID NO: 52 - protein, 15 aa, Artificial sequence
  CXCR4 binding peptide
MAASLIWRPAKAKKK

SEQ ID NO: 53 - protein, 15 aa, Artificial sequence
  CXCR4 binding peptide
MARSLIWRPARRRRR

SEQ ID NO: 54 - protein, 14 aa, Artificial sequence
  CXCR4 binding peptide
ARSLIXRLARRXRR

SEQ ID NO: 55 - protein, 14 aa, Artificial sequence
  CXCR4 binding peptide
ARHLIXRHARRXRR

SEQ ID NO: 56 - protein, 14 aa, Artificial sequence
  CXCR4 binding peptide
HRSLIXRHARRXRR

SEQ ID NO: 57 - protein, 9 aa, Artificial sequence
  CXCR4 binding peptide
XRHARRXRR

SEQ ID NO: 58 - protein, 14 aa, Artificial sequence
  CXCR4 binding peptide
ARSLIXRLARRXRR

SEQ ID NO: 59 - protein, 10 aa, Artificial sequence
  CXCR4 binding peptide
XXRHARRXRR

SEQ ID NO: 60 - protein, 14 aa, Artificial sequence
  CXCR4 binding peptide
ARSLIXRPARRXRR

SEQ ID NO: 61 - protein, 10 aa, Artificial sequence
  cyclic library peptide
MKXDHRXXKN

SEQ ID NO: 62 - protein, 8 aa, Artificial sequence
  Preferred peptide for PDZ binding domain
KKKKETDV

SEQ ID NO: 63 - protein, 12 aa, Artificial sequence
  Preferred peptide for Src binding domain
EPQYEEIPIYLK

SEQ ID NO: 64 - protein, 7 aa, Artificial sequence
  Preferred peptide for 14-3-3 binding domain
RLSHSLP

SEQ ID NO: 65 - protein, 5 aa, Artificial sequence
  Preferred peptide for SH2 binding domain
YEEIY

SEQ ID NO: 66 - protein, 6 aa, Artificial sequence
  Preferred peptide for SH3 binding domain
PXRPXR

SEQ ID NO: 67 - protein, 7 aa, Artificial sequence
  Preferred peptide for Lim binding domain
GPXGPXX

SEQ ID NO: 68 - protein, 14 aa, Artificial sequence
  Initial library
MAXXXXRXXXXKKK

SEQ ID NO: 69 - protein, 15 aa, Artificial sequence
  Library peptide
MAXXXXWXXXXAKKK

SEQ ID NO: 70 - protein, 15 aa, Artificial sequence
  Library peptide
MARXXIWRXXXAKKK

SEQ ID NO: 71 - protein, 14 aa, Artificial sequence
  Library peptide
MXXXXWXXXXAKKK

SEQ ID NO: 72 - protein, 14 aa, Artificial sequence
  CXCR4 binding peptide
ARSLIXRXARRXRR

SEQ ID NO: 73 - protein, 7 aa, Artificial sequence
  CXCR4 binding phage library peptide
PAHYPML

SEQ ID NO: 74 - protein, 7 aa, Artificial sequence
  CXCR4 binding phage library peptide
QYATPNK

SEQ ID NO: 75 - protein, 7 aa, Artificial sequence
  CRCR4 binding phage library peptide
QQRSTAF

SEQ ID NO: 76 - protein, 7 aa, Artificial sequence
  CRCR4 binding phage library peptide
PFRATTE

SEQ ID NO: 77 - protein, 7 aa, Artificial sequence
  CRCR4 binding phage library peptide
TDKLLLD

SEQ ID NO: 78 - protein, 7 aa, Artificial sequence
  CXCR4 binding phage library peptide
HTQHVRT

SEQ ID NO: 79 - protein, 7 aa, Artificial sequence
  CXCR4 binding phage library peptide
LGVKAPS

SEQ ID NO: 80 - protein, 7 aa, Artificial sequence
  CXCR4 binding phage library peptide
DLQARYS

SEQ ID NO: 81 - protein, 7 aa, Artificial sequence
  CXCR4 binding phage library peptide
SLTEPSL

SEQ ID NO: 82 - protein, 7 aa, Artificial sequence
  CXCR4 binding phage library peptide
STWPLAQ

SEQ ID NO: 83 - protein, 7 aa, Artificial sequence
  CXCR4 binding phage library peptide
RTTSDAL

SEQ ID NO: 84 - protein, 15 aa, Artificial sequence
  CXCR4 binding peptide
MARSLIWRPAEAKKK

SEQ ID NO: 85 - protein, 15 aa, Artificial sequence
  CXCR4 binding peptide
MARSLIWRPRKAKKK

SEQ ID NO: 86 - protein, 15 aa, Artificial sequence
  CXCR4 binding peptide
MARSLIERPAKAKKK

SEQ ID NO: 87 - protein, 15 aa, Artificial sequence
  CXCR4 binding peptide
MARSLIWRPAKAKQK

SEQ ID NO: 88 - protein, 15 aa, Artificial sequence
  CXCR4 binding peptide
MARSLIWRPAKALKK

SEQ ID NO: 89 - protein, 15 aa, Artificial sequence
  CXCR4 binding peptide
MARSLIWRPAKAKEK

SEQ ID NO: 90 - protein, 15 aa, Artificial sequence
  CXCR4 binding peptide
MARELIWRPAKAKKK

SEQ ID NO: 91 - protein, 15 aa, Artificial sequence
  CXCR4 binding peptide
MARSLLWRPAKAKKK

SEQ ID NO: 92 - protein, 15 aa, Artificial sequence
  CXCR4 binding peptide
MARSLIWRPAKLKKK

SEQ ID NO: 93 - protein, 15 aa, Artificial sequence
  CXCR4 binding peptide
MARSLIWRPAKAXKK

SEQ ID NO: 94 - protein, 15 aa, Artificial sequence
  CXCR4 binding peptide
XARSLIWRPAKAKKK

SEQ ID NO: 95 - protein, 15 aa, Artificial sequence
  CXCR4 binding peptide
MARSLIQRPAKAKKK

SEQ ID NO: 96 - protein, 15 aa, Artificial sequence
  CXCR4 binding peptide
MARSLIWRPAKAKKR

SEQ ID NO: 97 - protein, 15 aa, Artificial sequence
  CXCR4 binding peptide
MARSLIWRPAQAKKK

SEQ ID NO: 98 - protein, 15 aa, Artificial sequence
  CXCR4 binding peptide
MARSLIWRPAKAWKK

SEQ ID NO: 99 - protein, 14 aa, Artificial sequence
  CXCR4 binding peptide
ARSLIWRPAKAKKK

SEQ ID NO: 100 - protein, 15 aa, Artificial sequence
  CXCR4 binding peptide
MLRSLIWRPAKAKKK

SEQ ID NO: 101 - protein, 16 aa, Artificial sequence
  CXCR4 binding peptide
EMARKLIWRPAKAKKK

SEQ ID NO: 102 - protein, 10 aa, Artificial sequence
  CXCR4 binding peptide
XXRLARRXRR

SEQ ID NO: 103 - protein, 10 aa, Artificial sequence
  CXCR4 binding peptide
XXRPARKXRR

SEQ ID NO: 104 - protein, 9 aa, Artificial sequence
  CXCR4 binding peptide
XRPARRXRR

SEQ ID NO: 105 - protein, 10 aa, Artificial sequence
  CXCR4 binding peptide
XXRPRARXRR

SEQ ID NO: 106 - protein, 10 aa, Artificial sequence
  CXCR4 binding peptide
XXRPARRLRR

SEQ ID NO: 107 - protein, 10 aa, Artificial sequence
  CXCR4 binding peptide
XXRPARRXRR

SEQ ID NO: 108 - protein, 10 aa, Artificial sequence
  CXCR4 binding peptide
XXEPRAKLAK

SEQ ID NO: 109 - protein, 10 aa, Artificial sequence
  CXCR4 binding peptide
XXEPARRXRR

SEQ ID NO: 110 - protein, 10 aa, Artificial sequence
  CXCR4 binding peptide
XWRPARRXRR

SEQ ID NO: 111 - protein, 10 aa, Artificial sequence
  CXCR4 binding peptide
XXRPARRXRK

SEQ ID NO: 112 - protein, 10 aa, Artificial sequence
  CXCR4 binding peptide
XXRPARRXAR

SEQ ID NO: 113 - protein, 10 aa, Artificial sequence
  CXCR4 binding peptide
XXEPRAKLAK

SEQ ID NO: 114 - protein, 14 aa, Artificial sequence
  CXCR4 binding peptide
ARSLIXRXARRXRR

SEQ ID NO: 115 - protein, 14 aa, Artificial sequence
  CXCR4 binding peptide
ARSLIXRXARRXRR

SEQ ID NO: 116 - protein, 14 aa, Artificial sequence
  CXCR4 binding peptide
ARSLIXRXARRXRR

SEQ ID NO: 117 - protein, 14 aa, Artificial sequence
  CXCR4 binding peptide
ARSLIXRXARRXRR

SEQ ID NO: 118 - protein, 14 aa, Artificial sequence
  CXCR4 binding peptide
ARSLIXRXARRXRR

SEQ ID NO: 119 - protein, 14 aa, Artificial sequence
  CXCR4 binding peptide
ARSLIXRPRARXRR

SEQ ID NO: 120 - protein, 14 aa, Artificial sequence
  CXCR4 binding peptide
ARSLIXRLRARXRR

SEQ ID NO: 121 - protein, 14 aa, Artificial sequence
  CXCR4 binding peptide
ARSLIXRLARRLRR

SEQ ID NO: 122 - protein, 14 aa, Artificial sequence
  CXCR4 binding peptide
ARSLILRLARRXRR

SEQ ID NO: 123 - protein, 16 aa, Artificial sequence
  CXCR4 binding peptide
EMARKLIXRXARRXRR

SEQ ID NO: 124 - protein, 14 aa, Artificial sequence
  CXCR4 binding peptide
ARSLIXEXRAKXRR

SEQ ID NO: 125 - DNA, 1059 bp, Homo sapiens
  CDS (1)..(1059): human CXCR4 gene
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 126 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS